SEQUENCE LISTING (7 sequences)

SEQ ID NO: 1 - protein, 8 aa, Artificial Sequence
  Synthetic
DYKDDDDK

SEQ ID NO: 2 - protein, 9 aa, Artificial Sequence
  Synthetic
YPYDVPDYA

SEQ ID NO: 3 - protein, 10 aa, Artificial Sequence
  Synthetic
EQKLISEEDL

SEQ ID NO: 4 - protein, 15 aa, Artificial Sequence
  Synthetic
KETAAAKFERQHMDE

SEQ ID NO: 5 - protein, 8 aa, Artificial Sequence
  Synthetic
WSHPQFEK

SEQ ID NO: 6 - protein, 38 aa, Artificial Sequence
  Synthetic
MDEKTTGWRGGHVVEGLAGELEQLRARLEHHPQGQREP

SEQ ID NO: 7 - protein, 20 aa, Artificial Sequence
  Synthetic
GVMPREETDSKTASPWKSAR

(19) United States
(12) Patent Application Publication
(10) Pub. No.: US 2018/0284124 A1
(43) Pub. Date: Oct. 4, 2018

(54) METHOD FOR REDUCTIVE AND OXIDATIVE MASS LABELING

(71) Applicants: Michael Joseph Pugia, Ganger, IN (US); Zane Baird, Brigham City, UT (US); Zehui Cao, Carmel, IN (US)

(72) Inventors: Michael Joseph Pugia, Ganger, IN (US); Zane Baird, Brigham City, UT (US); Zehui Cao, Carmel, IN (US)

(21) Appl. No.: 15/942,557

(22) Filed: Apr. 1, 2018

Related U.S. Application Data
(60) Provisional application No. 62/480,369, filed on Apr. 1, 2017.

(51) Int. Cl. G01N 33/58; G01N 1/40

(52) U.S. Cl. CPC G01N 33/585; H01J 49/4225; G01N 1/40

(57) ABSTRACT

A method for capture and release of mass labels whereby a mass label is attached to rare molecule by electrochemical oxidation and released by electrochemical reduction, whereby, the mass label and rare molecule are removed from the sample by affinity binding and the mass label with or without rare molecule are released by electrochemical reduction. The method allows isolation and detection of one or more different populations of rare molecules in a sample suspected of containing one or more different populations of rare molecules and non-rare molecules. Bond breakage or bond formation of mass labels occurs by an electrochemical reaction in porous matrix placed between an anode and cathode electrode in a thin layer electrochemical reactor. Capture and release of mass labels by electrochemical reduction or oxidation wherein the chemical alteration agents are not used.

### BACKGROUND

The invention relates to methods for enriching and detecting rare molecules relative to non-rare molecules. In some aspects the invention relates to methods, apparatus and kits for detecting one or more different populations of rare molecules in a sample suspected of containing the one or more different populations of rare molecules and non-rare molecules. In some aspects, the invention relates to methods and kits for detecting one or more different populations of rare molecules that are freely circulating in samples. In other aspects, the invention relates to methods and kits for detecting one or more different populations of rare molecules that are associated with rare cells in a sample suspected of containing the one or more different populations of rare cells and non-rare cells.

The detection of rare molecules in the range of 1 to 50,000 copies (fentamolar (fM) or less) cannot be achieved by conventional affinity assays, which require a number of molecular copies far above the numbers found for rare molecules. For example, immunoassays cannot typically achieve a detection limit of 1 picomolar (pM) or less. Immunoassays are limited by the affinity binding constant of an antibody, which is typically not higher than 10⁻¹² (1 pM). Immunoassays require at least 100-fold antibody excess due to the off-rate being 10⁻¹³, and the solubility of the antibody protein limits driving the reaction to completion. As a typical sample volume is rarely greater than 10 μL, concentration of 1 pM requires 60 million copies of a rare molecule for detection, far greater than the range for a rare molecule. The detection of circulating proteins that are not cell bound is also desirable. This same issue of solubility of the antibody prevents conventional immunoassays from reaching sub-attomolar levels.

The detection of circulating rare molecules that are in the sample is complicated by being a mixture of rare and non-rare molecules. The materials can be cellular, e.g. internal to cells or “cell free” material and not bound or associated to any intact cell. Cell free rare molecules are important in medical applications such as, for example, diagnosis of cancer in tissues or other diseases. Here circulating rare molecules are shed from tissues into circulation and it is understood that cell free rare molecules correlate to the total amount of rare molecules in diseased tissues, for example tumor distributed throughout the body. Cell free analysis requires isolation and detection of free rare molecules from very small fraction of all free molecules in sample. When cell free molecules are shed into the peripheral blood from diseased cells in tissues, these molecules are mixed with molecules from normal cells. For example, approximately 10⁹ cells are present in a cubic cm of diseased tissue. If this tissue mass was fully dissolved into the 5 L of blood in the body this would only be 2 million cells per 10 mL of blood and still very rare, considering there are an average of 75 million leukocytes and 50 billion erythrocytes per 10 mL blood releasing non-rare molecules.

The detection of rare molecules that are cell bound or included in a cell is also important in medical applications such as, for example, diagnosis of many diseases that can be propagate from a single cell. The analysis of molecules of certain rare cells has extremely important medical applications, and requires isolation and detection of nucleic acids from very small fraction of cells in sample under analysis. For example, circulating tumor cells (“CTCs”) are of particular interest in the diagnosis of metastatic cancers. In conventional methods, CTC are isolated from a 10 mL whole blood sample by first removing red blood cells (RBCs) by lyses and leaving a few hundred CTCs mixed with about 800,000,000 white blood cells (“WBCs”). In second step, the sample can be filtered, so a few CTCs are mixed with about 15,000 WBC. Therefore purity of rare cell molecules is extremely impure and only 0.01% to 0.00001% even after enrichment steps.

Rare cells can be analyzed down to the single cell level by a conventional scanning microscopy after purification by filtration methods (Magbanua 2015). Antibodies with fluorescent labels can detect as few as 50,000 molecules at 1 attomolar (aM) for some proteins in a single cell. This is due to the extremely small sample detection volume (1 nanoliter (nL) or less) of a microscopic analysis volume of a single cell. Additionally, as few as 1,000 molecules (fM) can be detected with antibodies after enzyme amplification (50-fold amplification). Further, molecular analysis (in-situ hybridization) of cells can be done down to a single molecule level due to the higher affinity of nucleic acid probes. However, even with automation of the scanning and analysis, the microscopy method can take 24 hours or more for each sample to be scanned. Additionally, all the rare cells with multiple images must be examined visually by the pathologist to determine the significance of protein amounts measured.

Mass Spectrometry (MS) is an extremely sensitive and specific technique very well suited for detecting small molecules (about 300 daltons (Da)) and medium sized molecules (about 3000 Da) at pM concentrations in extremely small sample volumes (1 nanoliter (nL) or less). MS also has the ability to simultaneously measure hundreds of highly abundant components (multiplexing) present in complex biological media in a single assay without the need for labeled reagents. The method offers specificity until the biological complexity causes overlapping masses. Of the MS combined techniques (ionization and separation), triple quad mass spectrometry (MS-MS), liquid chromatography-tandem mass spectrometry (LC-MS/MS) is limited to small mass analytes and liquid chromatography-tandem mass spectrometry (LC-MS/MS) with multiple reaction monitoring (MRM) (LC-MRM-MS) is limited to high abundance proteins. In both cases the use of liquid chromatography makes automation difficult due to run times, cost, complexity and maintainability. Matrix-assisted laser desorption/ionization using a time-of-flight mass spectrometer (MALDI-TOF) combined technique is well suited for high sensitivity for low abundance molecules needed for rare molecular analysis; however, specificity for the biological media causes overlapping masses.

The current state of mass spectroscopy is not competitive with routine clinical diagnostics laboratory systems, with noted problems in the inability to separate markers of interest (sample preparation), loss of sensitivity due to background in clinical sample (picomolar (pM) reduced to nanomolar (nM)), the inability to routinely work with small nL sample volumes as samples less than 1 microliter (μ1), inefficient ionization of some markers without labeling and inefficiently separating masses from interfering peaks in complex samples such as blood (matrix over lapping peaks). In addition, MS often has an inability to detect certain masses due to competition with other mass of the same mass being ionized. These issues typically cause problems due to false results.

Another problem for mass spectral analysis is that to ionize mass readily for quantitation of results; the methods requires limiting analysis to detection to smaller masses of less than 3 kilodaltons (kDa) with atoms that can be charged and made into parent ions. Proteins are typically greater than 10 kDa to 1000 kDa and are more difficult to ionize as parent ions and thus quantitiate. To achieve quantitative mass spectral analysis, the proteins have to be broken into smaller fragments typically by proteolysis with enzymes like trypsin. However, the trypsinization reaction of proteins is not reproducible; not all proteins and bound forms can be fragmented; certain epitopes or forms of interest are fragmented and cannot be detected; and various components of the sample inhibit the activity of trypsin, for example. Another problem is that these fragments often do not relate to the clinical state as they are not the relevant molecule regions.

One approach to solve this problem, is to chemically add a mass label to the molecule to be measured (Demmer 2012). This mass labeling approach has been a helpful approach to detecting cells, tissues, peptides and proteins are detected by mass spectrometry. Chemical labeling works by introducing a known mass on the molecule to be measured by a chemical reaction. Rare molecule amines are typically reacted with compounds like succinimides to form amides linkages to mass labels. Alcohols are typically reacted with acyl and tosyl chlorides to form tosylates and ester linkages to mass labels. Dienes are typically reacted with dienophiles to form ring linkages to mass labels. Carbonyls are typically reacted hydroxyl amines to form with oximes linkages to mass labels. Examples of chemically adding a mass label to proteins or peptides are also known such as by acetylation or esterification reaction of sulfhydryl groups or amine groups on the amino acid residues, typically lysine, arginine or cysteine. Other approaches use trypsin, to catalyze the exchange of two ¹⁶O atoms for two ¹⁸O atoms in proteins or peptides to be measured. Carnitine moieties are also commonly used in mass label peptide analysis, with peptide chemically modified to carry the carnitine mass label. Dimethylation of peptides is another mass labeling approach. The Isotope-Coded Affinity Tags (ICAT) method is yet another example, adding a reactive polyether linker region with eight deuteriums to the cysteine of peptides.

Labeling with mass labels that can be cleaved is used in several new approaches, such as Tag for Relative and Absolute Quantitation (iTRAQ) (Ross et al., 2004) and Tandem Nass Tag (TMT) (Thompson et al., 2003) methods. These methods attach a mass label to an analyte to be detected via a cleavable linker arm, upon high energy collision dissociation (HCD), then the mass label is released and measured. The mixture of all peptides is labeled in such a way that all labeled peptides are isobaric and chromatographically indistinguishable. Only upon peptide fragmentation when the different mass tags are released can the peptides be distinguished. While this methods still suffer from contamination with multiple masses, it is not as limited by the mass of the analyte to be measured. While all these mass labeling approaches allow masses to be more easily ionized and unique identified, they all still suffer from contamination with multiple masses and limited by the mass of the analyte to be measured.

Therefore, other approaches were sought to avoid or reduce the contamination problems associated with these current mass spectral analysis methods. One common approach is affinity agents with mass spectrometry allows masses to be captured by an affinity agent and purified from contaminates prior to detection (Zhu 2006). This method uses an affinity agent, like an antibody, attached to a capture surface or particle. While this method has been successfully used for clinical measurement of renin, it still requires trypsinization reaction for measurement of other proteins and is still limited by the ionization of the molecule to be detected (Popp 2014). Also it appears that this method of sample preparation remains a difficult and complex multistep process to automate and is noncompetitive with other detection technologies used in the clinical laboratory.

Combining affinity agents and mass labeling with mass spectrometry is accomplished by adding mass labels directly to the affinity agent (Bandura 2009, Lee 2008). In this approach a metal, is chelated or attached to antibodies against rare cell molecules of interest. Either the metal itself acts as a mass labels and is released from the affinity agent during inductively coupled plasma time-of-flight mass spectroscopy or an additional release-able mass label is added to the metal. This approach allows detection of different populations of target rare molecules which bound to unique affinity agents. The Mass label corresponds to one of the populations of target rare molecules. The organic mass label can also be attached to a particle where a labeled particle is associated to capture particle and an affinity agent (Baird 2016). The advantage of the combined approach, is that this allows a common MS detection label for any analyte that can be bound to any affinity agent. However, using metals for Mass labels can be difficult to quantitate as internal standards which ionize identically to the mass label are not often possible and detection of metals requires inductively coupled plasma and the release of mass labels.

In Pugia PCT/US2015/033278 the metal is replaced with an mass label on an organic nanoparticle. Here a chemical is still used as an “alteration agent” to release the mass label from the affinity agent by breaking a disulfide bond. The mass label contains a quaternary ammonium group and is connected by a cleavable linkage, for example a disulfide from a cysteine on the mass label to an affinity agent using conjugation with N-succinimidyl 3-(2-pyridyldithio)-propionate) (SPDP). A reducing agent namely dithiothreitol (DTT) or tris(2-carboxyethyl) phosphine (TCEP) agent” is used to break the disulfide bond. Other researchers replace the mass label with a quaternary imidazolium, phosphonium, or sulfonium organic molecules and released from the affinity agent by cleaving a acetal or ketal bond with strong acid. Later Chen on 2016 adds a quaternary amine directly to an affinity agent through and ester bond and uses bases as chemical alteration agents to release the mass label.

Chemical reaction for S—S bond formation and breakage are well known and rely on oxidation and reduction reactions using alteration agents and catalysts. For example, thiol reactant can be oxidized to disulfides by a sulfoxide reactant in the presence of a halogen-hydrogen halide catalyst. The catalysts for the reaction are iodine, hydrogen iodide, bromine, hydrogen bromide, chlorine, hydrogen chloride and mixtures thereof (Lowe in U.S. Pat. No. 3,954,800 1976, Tamm 1991). Disulfide reactant can be reduced to thiols by a phosphine, cupric, sodium borohydride, and metallic zinc reactant, as well as by exchange reactions with other thiols, and by electrochemical reduction (Thorpe 2014). Thiol also are also known to reversibility bind to metals like gold, cobalt, gold iron, and zinc (Jacob 2003). This metal binding ability has been used for affinity tagging for protein and peptide isolation (Pasker 2006) where a S-metal bond formation rely on oxidation reactions. This principle of mass tagging for affinity purification is also well applied to S-metal, N-metal and O-metal bonds like thiol and zinc, histidine and cobalt and gamma carboxyglutamates and cobalt (Hale 1995, Arnold 1995).

However, all these approaches either rely on chemical alteration agent to release or attach the mass label or are too slow to be effective for rapid analysis take minutes to complete. The alteration agent can speed up reactions but these chemicals have several key disadvantages, namely they can suppress ionization, the released mass label groups in a reactive state which re-bind to sample or assay component, or degrade the mass label all causing a loss of sensitivity. The time to release the mass label can be several minutes long, and the materials can be prone to spontaneous cleavage by actions of chemicals in sample or reagents in the assay. For example, using a simple acid can release the mass label, but these mass labels will suffer from instability due to premature hydrolysis in most aqueous buffers needed for biological assays.

The field still requires an improved release and formation mechanism for mass labels which is are stable, have faster time to release or form, use no reagents or non-interfering reagent and minimize loss. Methods that allow release of mass labels from affinity agents or rare molecules without the use of the chemical alteration agents are desired. There is, therefore, a long felt need to develop methods and apparatus that provide for specific or selective mass labeling and rapid release of mass labels into a mass spectrometer while avoiding reduced sensitivity.

### SUMMARY OF THE INVENTION

The invention is a method for the attachment or release of mass labels, said method comprising attaching or releasing said mass labels by electrochemical reduction or electrochemical oxidation.

In accordance with the invention, we provide methods of attachment or release of mass labels by electrochemical reduction or oxidation by breaking or forming an —X—Y— bond where X and Y can be a S, O, C, N or a metal whereby chemical alteration agents are not used. The method allows isolation and detection of one or more different populations of rare molecules in a sample suspected of containing one or more different populations of rare molecules and non-rare molecules

Some examples in accordance with the invention described herein are directed to, a method of bond breakage or bond formation of mass labels by an electrochemical reaction in a porous matrix placed between an anode and cathode electrode in a thin layer electrochemical reactor. Capture and release of mass labels by electrochemical reduction or oxidation whereby chemical alteration agents are not used in such a way as to interfere with mass analysis.

Some examples in accordance with the invention described herein are directed to, a method of capture and release of mass labels whereby a mass label is attached to an affinity agent by an X—Y bond, the mass label and rare molecule are removed from the sample by a affinity agent, the mass labels are released by electrochemical reduction breaking of the X—Y bond and the mass label measured and related to rare molecules.

Some examples in accordance with the invention described herein are directed to, a method of capture and release of mass labels whereby a mass label is attached to rare molecule by electrochemical oxidation, the mass label and rare molecule are removed from the sample by affinity agent and mass label or rare molecule are released by electrochemical reduction.

### BRIEF DESCRIPTION OF THE DRAWINGS

The drawings provided herein are not to scale and are provided for the purpose of facilitating the understanding of certain examples in accordance with the principles described herein and are provided by way of illustration and not limitation on the scope of the appended claims.

FIGS. 1a and 1b show a schematic depicting an example of a method in accordance with the invention for detection of a rare molecule whereby a mass label is attached to affinity agent that binds a rare molecule. As shown in FIG. 1a, the rare molecule 2 is cell bound. The attachment is shown through a labeled particle 1 and the cleavable linkage X—Y shown as 3 between labeled particle 1 and mass labels 4 and the affinity agent 5 is bound directly to the label particle 1. In FIG. 1b, the cell 6 bound rare molecule 2 are separated onto a porous matrix 7 with pores 8. The labeled particle 1 with mass labels 4 are bound to rare molecule cell 6 on porous matrix 7 through the affinity agent 5, whereby unbound mass label particles are washed though the porous matrix 7.

FIGS. 2a and 2b are a schematic depicting an example of a method in accordance with the invention described herein for detection of a rare molecule whereby a mass label is attached to an affinity agent that binds a rare molecule. In FIG. 2a the rare molecule 10 is cell free. The attachment is shown through a labeled particle 9 and the cleavable linkage 11 (X—Y) between labeled particle 9 and mass labels 12 and the affinity agent 13 is bound directly to the labeled particle 9. The rare molecule 10 is additionally bound to a second affinity agent 15 which is bound directly the capture particle 14. As shown in FIG. 2b, the bound rare molecule 16 is separated onto a porous matrix 17 having pores 18. The labeled particle 9 with mass labels 12 and capture particles 14 are bound to rare molecule 16 through the affinity agent 15 and captured on porous matrix 17, whereby unbound mass label particles are washed through the porous matrix 17.

FIGS. 3a and 3b is an additional schematic depicting an example of a method in accordance with the invention described herein directed to a method of capture and release of mass labels whereby a mass label is attached to rare molecule by electrochemical oxidation. In this case the rare molecule is cell free. In FIG. 3a, the attachment is shown through a cleavable linkage 19 (X—Y) between rare molecule 20 and mass label 21. The rare molecules 20 with mass labels 21 are bound to capture particles 22 through the affinity agent 23. In FIG. 3b, the bound rare molecule with mass label 24 is separated onto a porous matrix 25 having pores 26 using an affinity agent 27 which is bound directly to a capture particle 28. The rare molecules with mass labels 24 are bound to capture particles 28 through the affinity agent 27 and captured on porous matrix 25, whereby molecules not bound the affinity agent are washed though the porous matrix. After the mass label and rare molecule are removed from the sample with affinity agent, the rare molecule or mass label are released by electrochemical reduction.

FIGS. 4a and 4b illustrates another example of a method in accordance with the invention described herein directed to a method of capture and release of mass labels whereby a mass label is attached to rare molecule by electrochemical oxidation. In this case the rare molecule is cell free. In FIG. 4a, the attachment is shown through a labeled particle 29 and the cleavable linkage 30 (X—Y) between rare molecule 31 and mass label 32 on particle 29. The rare molecule 31 is additionally bound to a second affinity agent 33 which is bound directly to the capture particle 34. As shown in FIG. 4b, the rare molecules 31 with mass labels 32 are bound to capture particles 34 through the affinity agent 33 and captured on porous matrix 35 having pores 36, whereby molecules not bound the affinity agent are washed through the porous matrix. After the mass label and rare molecule are removed from the affinity agent, the rare molecules are released from the label particle by electrochemical reduction.

FIGS. 5a and 5b further show an example of a method in accordance with the invention described herein directed to a method of capture and release of mass labels whereby a mass label is attached to rare molecule by electrochemical oxidation. In this case the rare molecule is cell bound. In FIG. 5a, the labeled particle 37 is shown with the attachment of the cleavable linkage 38 (X—Y) between rare molecule 39 and mass label 40. Additionally, an affinity tag 41 is attached to the label particles 37. After the cells are lyzed and as shown in FIG. 5b, the affinity tag 41 is additionally bound to a second affinity agent 42 which is bound directly the capture particle 43. The rare molecules 39 with mass labels 40 are bound to capture particles 43 through the affinity agent 42 and captured on porous matrix 44 having pores 45, whereby molecules not bound to the affinity agent are washed though the porous matrix. After the mass label and rare molecule are removed from the affinity agent, the rare molecules are released from the labeled particle by electrochemical reduction.

FIG. 6 is another schematic depicting an example of a method in accordance with the invention described herein for a method where bond breakage or bond formation of mass labels occurs by an electrochemical reaction in a porous matrix placed between an anode and cathode electrode in a thin layer electrochemical reactor. In this example attachment or release of mass labels by electrochemical reduction or oxidation by breaking or a forming an —X—Y— bond where X and Y can be a S, O, C, N or metal. In the case of reduction, the mass label is released from rare molecule by cleaving the —X—Y— bond. The affinity agent can additionally be bound directly the label particle and the cleavable —X—Y— bond to the particle. In the case of oxidation, the mass label is attached to a rare molecule with a cleavable —X—Y— bond. The rare molecule can additionally be bound directly the label particle and the cleavable —X—Y— bond to the particle.

### DETAILED DESCRIPTION OF THE INVENTION

Methods, apparatus and kits in accordance with the invention described herein have application in any situation where detection or isolation of rare molecules is required. Examples of such applications include, by way of illustration and not limitation, methods of isolation and detection of rare molecules and modification with mass labels and method of affinity assay where mass label indicate binding of rare molecules to affinity agents.

In accordance with the invention described herein a bond breakage or bond formation of mass labels occurs by an electrochemical reaction in a porous matrix placed between an anode and cathode electrode in a thin layer electrochemical reactor.

Some examples in accordance with the invention described herein are directed to methods of detecting one or more different populations of rare molecules in a sample suspected of containing the one or more different populations of rare molecules and non-rare molecules through the detection of mass labels attached or bound to an affinity agent which are modified by an electrochemical reaction.

Some examples in accordance with the invention are directed at the detection or isolation of rare molecules where the rare molecules are modified by electrochemical reaction in a porous matrix placed between an anode and cathode electrode in a thin layer electrochemical reactor.

Some examples in accordance with the invention described herein are directed to methods of binding and separation of rare molecules where particles and cell are isolated on porous matrix and bound materials are retained for analysis.

Some examples in accordance with the invention are directed at the detection or isolation of rare molecules that are cell free while other examples are directed at the detection or isolation of rare molecules that are cell bound. Other examples are directed at the detection or isolation of rare molecules that are cell free.

Some examples in accordance with the invention described herein are directed to methods of electrochemical release of mass label from the rare molecule by reduction. While other examples in accordance with the principles described herein are directed to methods of electrochemical attachment of mass label to the rare molecule by oxidation.

Other examples in accordance with the invention described herein are directed to methods of selective detection of analytes by amplification of mass labels through attachment to nanoparticles labels. Some examples in accordance with the invention described herein are directed to methods of amplification where multiple mass labels are attachment to labeled particles. Additionally, the affinity agents can be linked to capture particles and capture particle isolated on a porous membrane.

Examples in accordance with the invention described herein are directed to methods and kits for peptide, protein or nucleic acid or analysis. Other examples in accordance with the principles described herein are directed to an apparatus for analysis.

The term “mass label” refers to a molecule having unique mass that corresponds to, and is used to determine a presence and/or amount of rare molecules or as affinity tag for rare molecules. Mass label can be attached to an affinity agent for a rare molecule, to a rare molecule, or to label particle. Additionally, the mass label can be released from an affinity agent, an rare molecule, or a label particle.

The term “electrochemical reaction” refers to an oxidation or reduction reaction where bond breakage or bond formation of mass labels occurs by an electrochemical reaction in a porous matrix placed between an anode and cathode electrode in a thin layer electrochemical reactior. Attachment occur by “electrochemical oxidation” forming a bond between atoms (X—Y) of two molecules. Release occurs by “electrochemical reduction” by breakage of a bond between atoms (X—Y) of two molecules.

The term “label particle” refers to a particle bound to a mass label agent. This particle can additionally be bound to an affinity agent or affinity tags.

The term “capture particle” refers to a particle attached to an affinity agent.

The term “affinity agent” refers to a molecule capable of selectively binding to a rare molecule of interest. This can be by directly binding the rare molecule, the mass label or attached molecules or particles. Affinity agent can be on a capture particle and can have an affinity for the mass label, rare molecule or affinity tag on label particle.

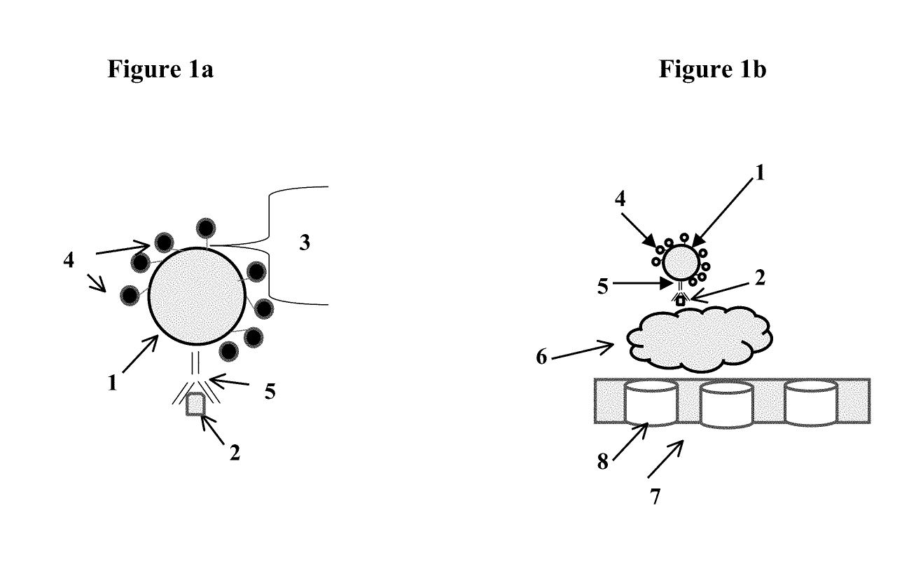

FIGS. 1a and 1b show a schematic depicting an example of a method in accordance with the invention for detection of a rare molecule whereby a mass label is attached to affinity agent that binds a rare molecule. As shown in FIG. 1a, the rare molecule 2 is cell bound. The attachment is shown through a labeled particle 1 and the cleavable linkage X—Y shown as 3 between labeled particle 1 and mass labels 4 and the affinity agent 5 is bound directly to the label particle 1. In FIG. 1b, the cell 6 bound rare molecule 2 are separated onto a porous matrix 7 with pores 8. The labeled particle 1 with mass labels 4 are bound to rare molecule cell 6 on porous matrix 7 through the affinity agent 5, whereby unbound mass label particles are washed though the porous matrix 7.

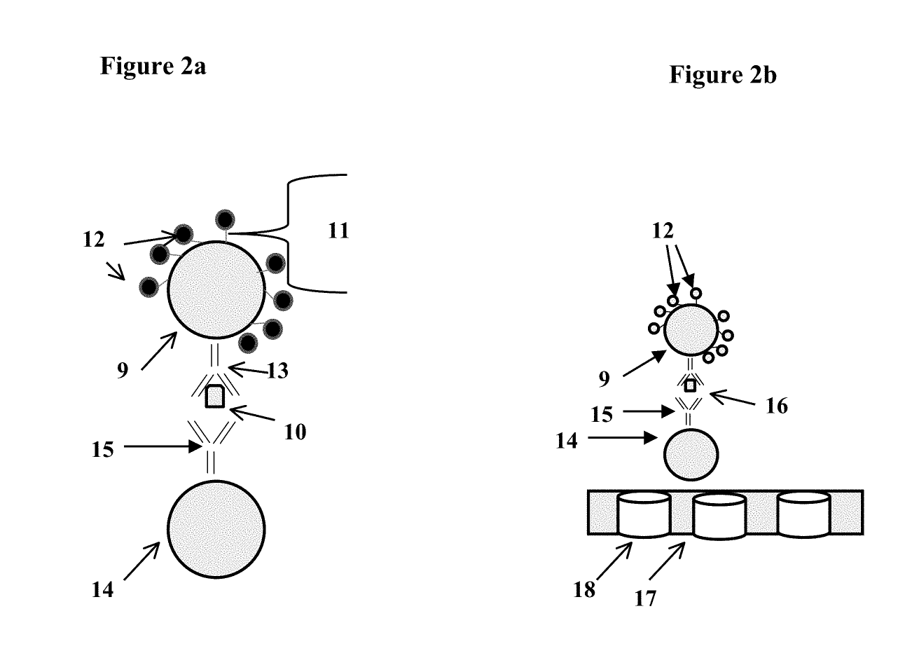

FIGS. 2a and 2b are a schematic depicting an example of a method in accordance with the invention described herein for detection of a rare molecule whereby a mass label is attached to an affinity agent that binds a rare molecule. In FIG. 2a the rare molecule 10 is cell free. The attachment is shown through a labeled particle 9 and the cleavable linkage 11 (X—Y) between labeled particle 9 and mass labels 12 and the affinity agent 13 is bound directly to the labeled particle 9. The rare molecule 10 is additionally bound to a second affinity agent 15 which is bound directly the capture particle 14. As shown in FIG. 2b, the bound rare molecule 16 is separated onto a porous matrix 17 having pores 18. The labeled particle 9 with mass labels 12 and capture particles 14 are bound to rare molecule 16 through the affinity agent 15 and captured on porous matrix 17, whereby unbound mass label particles are washed through the porous matrix 17.

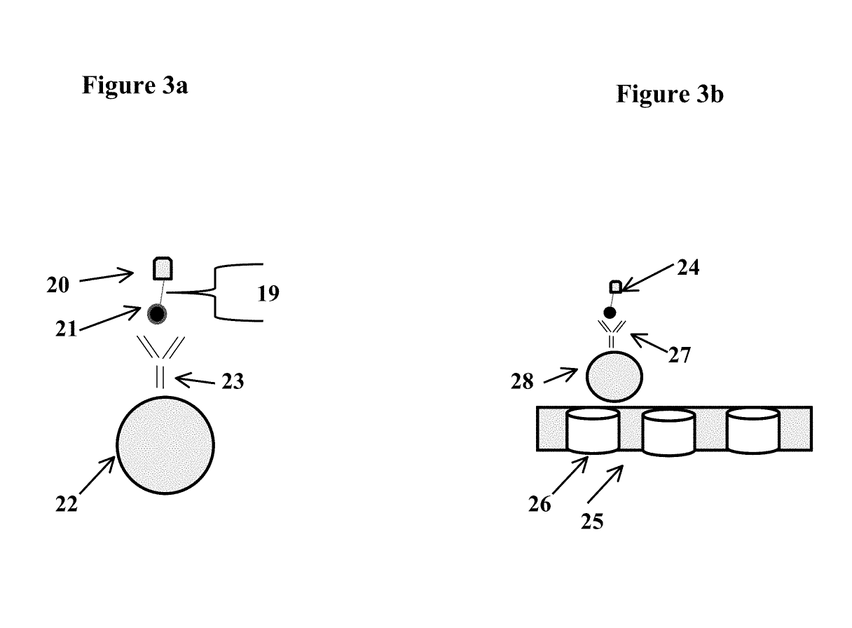

FIGS. 3a and 3b is an additional schematic depicting an example of a method in accordance with the invention described herein directed to a method of capture and release of mass labels whereby a mass label is attached to rare molecule by electrochemical oxidation. In this case the rare molecule is cell free. In FIG. 3a, the attachment is shown through a cleavable linkage 19 (X—Y) between rare molecule 20 and mass label 21. The rare molecules 20 with mass labels 21 are bound to capture particles 22 through the affinity agent 23. In FIG. 3b, the bound rare molecule with mass label 24 is separated onto a porous matrix 25 having pores 26 using an affinity agent 27 which is bound directly to a capture particle 28. The rare molecules with mass labels 24 are bound to capture particles 28 through the affinity agent 27 and captured on porous matrix 25, whereby molecules not bound the affinity agent are washed though the porous matrix. After the mass label and rare molecule are removed from the sample with affinity agent, the rare molecule or mass label are released by electrochemical reduction.

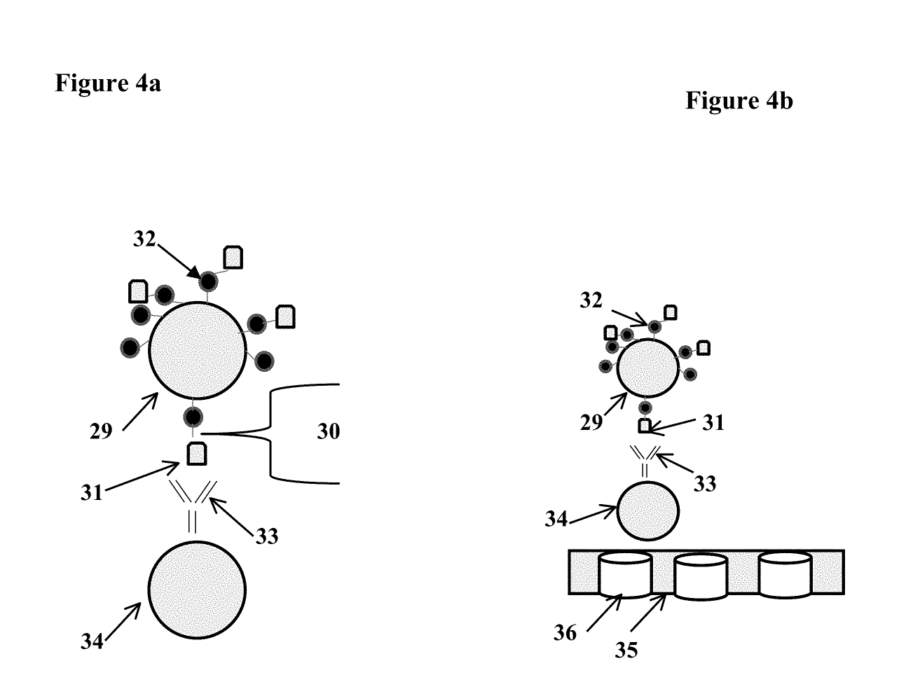

FIGS. 4a and 4b illustrates another example of a method in accordance with the invention described herein directed to a method of capture and release of mass labels whereby a mass label is attached to rare molecule by electrochemical oxidation. In this case the rare molecule is cell free. In FIG. 4a, the attachment is shown through a labeled particle 29 and the cleavable linkage 30 (X—Y) between rare molecule 31 and mass label 32 on particle 29. The rare molecule 31 is additionally bound to a second affinity agent 33 which is bound directly to the capture particle 34. As shown in FIG. 4b, the rare molecules 31 with mass labels 32 are bound to capture particles 34 through the affinity agent 33 and captured on porous matrix 35 having pores 36, whereby molecules not bound the affinity agent are washed through the porous matrix. After the mass label and rare molecule are removed from the affinity agent, the rare molecules are released from the label particle by electrochemical reduction.

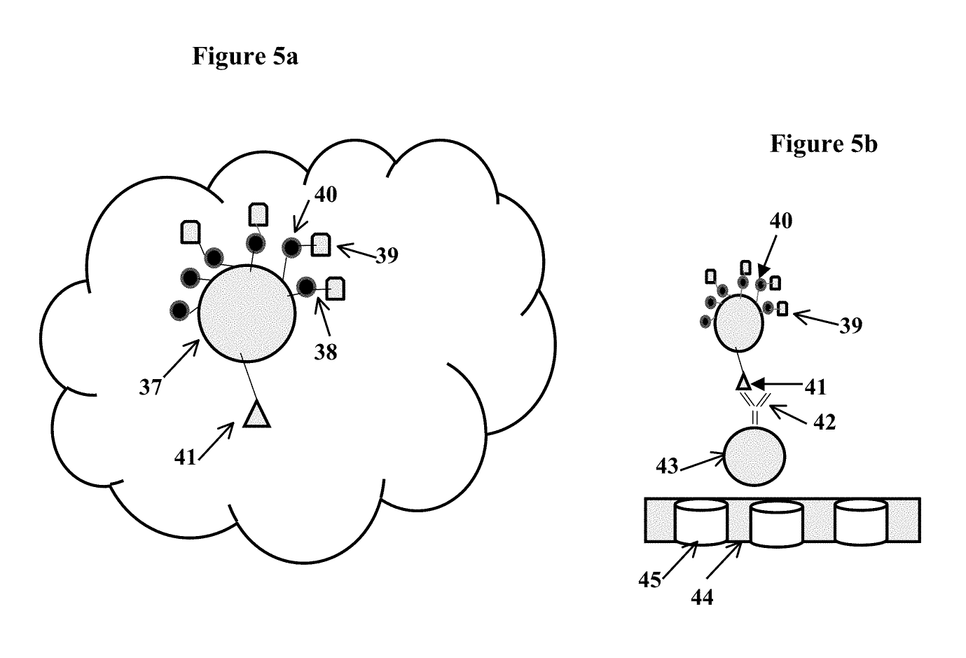

FIGS. 5a and 5b further show an example of a method in accordance with the invention described herein directed to a method of capture and release of mass labels whereby a mass label is attached to rare molecule by electrochemical oxidation. In this case the rare molecule is cell bound. In FIG. 5a, the labeled particle 37 is shown with the attachment of the cleavable linkage 38 (X—Y) between rare molecule 39 and mass label 40. Additionally, an affinity tag 41 is attached to the label particles 37. After the cells are lyzed and as shown in FIG. 5b, the affinity tag 41 is additionally bound to a second affinity agent 42 which is bound directly the capture particle 43. The rare molecules 39 with mass labels 40 are bound to capture particles 43 through the affinity agent 42 and captured on porous matrix 44 having pores 45, whereby molecules not bound to the affinity agent are washed though the porous matrix. After the mass label and rare molecule are removed from the affinity agent, the rare molecules are released from the labeled particle by electrochemical reduction.

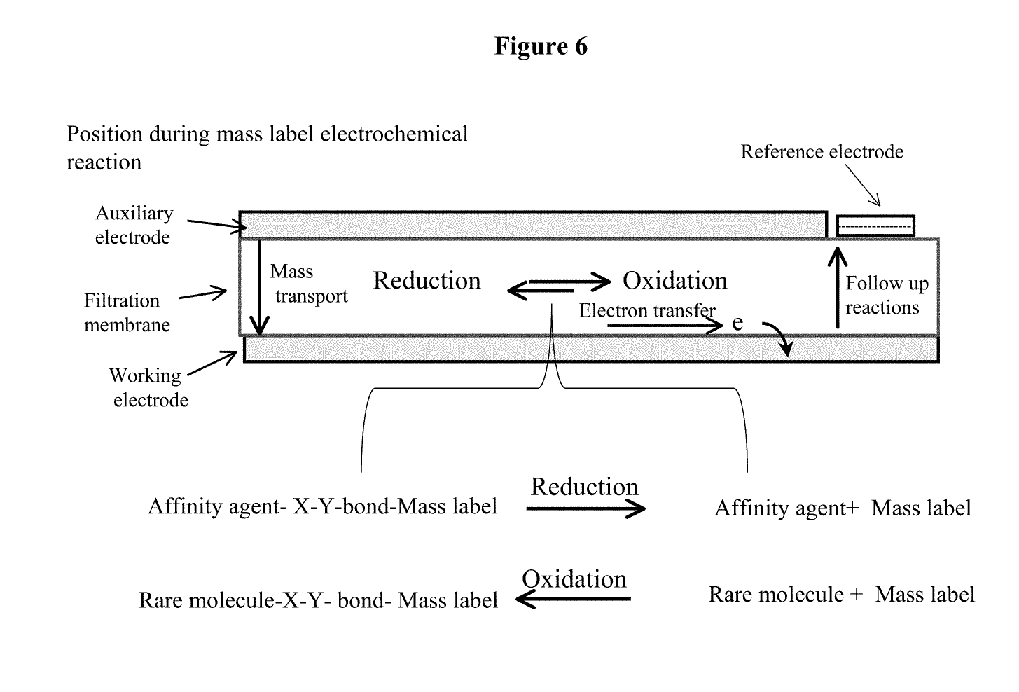

Another example of a method for detection or isolation of rare molecules are where the rare molecule or affinity agent for the rare molecule are modified by electrochemical reaction in porous matrix placed between an anode and cathode electrode in a thin layer electrochemical reactor as depicted in FIG. 6. The schematic depicts an example of a method in accordance with the invention for a method where bond breakage or bound formation of mass labels occurs by an electrochemical reaction in a porous matrix placed between an anode and cathode electrode in a thin layer electrochemical reactor. In this example attachment or release of mass labels by electrochemical reduction or oxidation is by breaking or forming an —X—Y— bond where X and Y can be a S, O, C, N or metal. In the case of reduction, the mass label is released from rare molecule by cleaving the —X—Y— bond. The affinity agent can additional be bound directly to the label particle and the cleavable —X—Y— bond to the particle. In the case of oxidation, the mass label is attached to rare molecule with a cleavable —X—Y— bond. The rare molecule can additional be bound directly the label particle and the cleavable —X—Y— bond to the particle.

### Examples of X—Y Bonds

In accordance with the invention, the mass label can be oxidatively attached to an affinity agent, rare molecule, or label particle by an —X—Y— bond where X and Y can be a S, N, O, C, or metal where the metal is Mn, Fe, Co, Ni, Cu, Zn, Mo, Tc, Ru Rh, Pd, Ag Cd, In, Sn, Ir, Pt Au, Hg, Ti or Pb. Additionally, the mass label can be reductively released from an affinity agent, a rare molecule, or a label particle by breaking—X—Y— bond where X and Y can be a S, O, C, N or metal where the metal is Mn, Fe, Co, Ni, Cu, Zn, Mo, Tc, Ru Rh, Pd, Ag Cd, In, Sn, Ir, Pt Au, Hg, Ti or Pb

Any “electrochemical reaction” can alter the mass label by bond breaking or bond formation to form a neutral, negative or positive ion by addition or removal of atoms, charges or electrons. This electrochemical reaction can result in a release of mass label or in attachment of a mass label. These electrochemical reactions can occur by reaction with a moiety, by derivatization, or by addition or by subtraction of molecules, charges or atoms, for example, or a combination of two or more molecules.

By way of illustration and not limitation, moiety examples of —X—Y— bond include S, N, O, C which are connected as parts of disulfides, thioethers, thioesters β-sulfones, sulfonyls, amides, esters, ethers, acetals, ketals, diazo, oxime, carbonates hydrazine, and peptides to name a few. Other examples of —X—Y— bond include examples where X is S, N, O, such a thiols, cysteine, histidine, arginine or tyrosine and Y is metal.

Examples can include moieties which facilitate bound breakage by connecting to either the X or Y such as a connected α-carbon which is activated, e.g., with a carbonyl, nitro or other electron withdrawing group; or where either the X or Y is connected to an aromatic e.g., such as phenol, quinone, imidazole, pyridine or other conjugated group; or where either the X or Y is in a amide cleavage site, including polypeptides connected to amino acids such as asparagine, tyrosine, tryptophan, carboxyglutamates and glutamine which undergo deamination.

Examples can including moieties which facilitate bond formation connecting X or Y such as a nucleophile-cleavable sites e.g., phthalamide, tosylate, cyclic anhydrides like succinic anhydride, cyclic imide anhydride like hydroxysuccinimide (NHS), N-heterocyclic carbenes; or where either the X or Y is a connected to an activated amine that promote formation of diazoniums, hydrazines, hydrazones, azo compounds, such as amines, imines, and aryl, benzyl or other conjugated group; or where either the X or Y is a connected to an activated carbonyl amine that promote formation of a oxime, including tosyl amines, aldhehydes; or where either the X or Y is a connected to an activated oxygen that promote formation of a C—O bond including acyl chlorides, acid anhydrides, carboylate ester, and sulfonate esters.

Electrochemical oxidation (attachment of mass label moieties) or electrochemical reduction (de-attachment of mass label moieties) may be additionally promoted by changing the ionic strength of the medium, adding a disruptive ionic substance, lowering or raising the pH, electron transfer, and enhanced by adding a surfactant, sonication, adding charged chemicals; or desalting solutions.

### Examples of Mass Labels

The phrase “mass label” refers to a group of molecules having unique masses. Mass labels are molecules of defined mass and include, but are not limited to, polypeptides, polymers, aromatic hydrocarbons, aliphatic fatty acids, proteins, metals, carbohydrates, organic amines, nucleic acids, and organic alcohols, for example, whose mass can be varied by substitution and chain size, for example. In the case of polymeric materials, the number of repeating units is adjusted such that the mass is in a region that does not overlap with a background mass from the sample. Mass label also cane generate a unique mass pattern due to structure and fragmentation upon ionization.

In cases of using mass labels for detection of rare molecules, the mass labels are typically below 3 kDA and each have unique mass that corresponds to, and is used to determine a presence and/or amount of, each different population of target rare molecules. The mass label is linked to either an affinity agent capable of binding a rare molecule or directly to a rare molecule. An electrochemical reduction reaction releases the mass label by bond breaking to form a neutral, negative or positive ion by breaking the bond between X—Y atoms. The mass labels can include ionized groups, such as quaternary ammonium salts like carnitine derivatives, quaternary aromatic ammonium salts like imidazole, pyrrole, histidine, quinoline, pyridine, indole, purine pyrimidine, and the like; tetra alkyl ammonium ions, tri alkyl sulfonium ions, tetra alkyl phosphonium ions and other examples.

In cases of using a mass label for isolation of rare molecules, the mass labels are used as affinity tags for rare molecules. In cases of using a mass label for isolation of rare molecules, the mass labels are used as affinity tags for rare molecules. Affinity tags are molecules which are capable of binding affinity agents or rare molecules. Affinity agents include metals, antibodies, proteins, and carbohydrate. Rare molecules include peptides and proteins. For example, the mass label affinity tags can be a metal binding molecule which binds and is included or added to the rare molecule. For example, the mass label affinity tags can be thiols groups added to the rare molecule. In other examples, the mass label affinity tags can be peptides, polypeptides or proteins which binds a metal, e.g. but not limited to proteins, peptides or molecules containing cysteine, histidine, arginine or tyrosine or thiol groups such as polyhistidine tags, polyarginine tags, glutathione S-transferase (GST tag), immunoglobulin or many others. In other examples, the mass label affinity tags can be a molecules which binds an antibody, e.g. but not limited to FLAG polypeptide tag sequence (SEQ ID NO:1 DYKDDDDK), influenza hemagglutinin (HA) polypeptide tag sequence (SEQ ID NO:2 YPYDVPDYA), c-Myc polypeptide tag sequence (SEQ ID NO:3 EQKLISEEDL), S-tag polypeptide tag sequence (SEQ ID NO:4 KETAAAKFERQHMDE), a puromycin which covalently links to a translated peptide or other molecules which are bound by serving as binding partners for antibody. In other examples mass label affinity tags can be a molecules which binds protein that are not antibody e.g. but not limited to biotin, strep II tag peptide sequence (SEQ ID NO:5 WSHPQFEK) which bind streptavidin-tactin protein, streptavidin-binding (SBP) peptide tag sequence (SEQ ID NO:6 MDEKTTGWRG GHVVEGLAGE LEQLRARLEH HPQGQREP) which bind streptavidin protein, calmodulin-binding peptide (CBP) sequence (SEQ ID NO:7 GVMPREETDSKT-ASPWKSAR) which bind calmodulin. In other examples mass label affinity tags can be a molecule that binds a carbohydrate like maltose-binding protein (MBP) (396 amino acid residues) that binds to amylose. Additionally, these mass tags polypeptides can be easily fused to recombinant proteins during sub cloning of its cDNA or gene expression using various vectors for various host organisms (E. coli, yeast, insect, and mammalian cells). Additionally the mass tags can add properties to the rare molecule e.g. MBP and S-tag affinity tags increase the solubility of protein rare molecule and FLAG peptide tag can be cleaved with a specific protease, e.g. enterokinase (enteropeptidase).

The nature of the mass label is dependent on one or more of the nature of the MS method employed, the nature of the MS detector employed, the nature of the target rare molecules, the nature of the affinity agent, nature of the capture particle, the nature of the label particle, the nature of any immunoassay employed, the nature of the sample, the nature of any buffer employed, the nature of the separation, for example. In some examples, the mass label can be varied by substitution and/or chain size. The mass labels produced from molecules of defined mass, and should not be present in the sample to be analyzed. Furthermore, the mass labels should be in the range detected by the MS detector, should not have over-lapping masses and should be detectable by primary mass.

Examples, by way of illustration and not limitation, of Mass label for use in methods in accordance with the principles described herein to produce Mass labels include, by way of illustration and not limitation, polypeptides, organic and inorganic polymers, fatty acids, carbohydrates, cyclic hydrocarbons, aliphatic hydrocarbons, aromatic hydrocarbons, organic carboxylic acids, organic amines, nucleic acids, organic alcohols (e.g., alkyl alcohols, acyl alcohols, phenols, polyols (e.g., glycols), thiols, epoxides, primary, secondary and tertiary amines, indoles, tertiary and quaternary ammonium compounds, amino alcohols, amino thiols, phenolic amines, indole carboxylic acids, phenolic acids, vinylogous acid, carboxylic acid esters, phosphate esters, carboxylic acid amides, carboxylic acids from polyamides and polyesters, hydrazone, oxime, trimethylsilyl enol ether, acetal, ketal, carbamates, ureas, guanidines, isocyanates, sulfonic acids, sulfonamides, sulfonylureas, sulfates esters, monoglycerides, glycerol ethers, sphingosine bases, ceramines, cerebrosides, steroids, prostaglandins, carbohydrates, nucleosides and therapeutic drugs, for example.

With polypeptide mass labels, for example, the chain length of the polypeptide can be adjusted to yield an Mass label in a mass region without background peaks. Furthermore, Mass labels may be produced from the Mass label precursors having unique masses, which are not present in the sample tested. The polypeptide Mass label precursors can comprise additional amino acids or derivatized amino acids, which allows methods in accordance with the principles described herein to be multiplexed to obtain more than one result at a time. Examples of polypeptide Mass label precursors include, but are not limited to, polyglycine, polyalanine, polyserine, polythreonine, polycysteine, polyvaline, polyleucine, polyisoleucine, poly-methionine, polyproline, polyphenylalanine, polytyrosine, polytryptophan, polyaspartic acid, polyglutamic acid, polyasparagine, polyglutamine, polyhistidine, polylysine and polyarginine, for example. Polypeptide Mass label precursors differentiated by mixtures of amino acids or derivatized amino acids generate masses having even or odd election ion with or without radicals. In some examples, polypeptides are able to be modified by catalysis.

In some examples in accordance with the principles described herein, the Mass label can comprise an isotope such as, but not limited to, ²H, ¹³C, and ¹⁸O, for example, which remains in the Mass label that is derived from the Mass label. The Mass label can be detected by the primary mass or a secondary mass after ionization. In some examples, the Mass label is one that has a relatively high potential to cause a bond cleavage such as, but not limited to, alkylated amines, acetals, primary amines and amides, for example, where the Mass label can generate a mass that has even or odd election ion with or without radicals. Selection of the polypeptide can generate a unique MS spectral signature.

Internal standards are an important aspect of mass spectral analysis. In some examples, a second mass label can be added that can be measured (as an internal standard) in addition to the Mass label used for detection of the rare target molecule. The internal standard has a similar structure to the mass label with a slight shift in mass. The internal standards can be prepared that comprise additional amino acids or derivatized amino acids. Alternatively, the internal standard can be prepared by incorporating an isotopic label such as, but not limited to ²H (D), ¹³C, and ¹⁸O, for example. The MS isotope label has a mass higher than the naturally-occurring substance. For example, the isotope labeled Mass labels, for example, glycerol-C-d7, sodium acetate-C-d7, sodium pyruvate-C-d7, D-glucose-C-d7, deuterated glucose, and dextrose-C-d7, would serve as internal standards for glycerol, sodium acetate, sodium pyruvate, glucose and dextrose, respectively.

MS analysis determines the mass-to-charge ratio (m/z) of molecules for accurate identification and measurement. The MS method ionizes molecules into masses as particles by several techniques that include, but are not limited to, matrix-assisted laser desorption ionization (MALDI), atmospheric pressure chemical ionization (APCI), electrospray ionization (ESI), inductive electrospray ionization (iESI), chemical ionization (CI), and electron ionization (EI), fast atom bombardment (FAB), field desorption/field ionization (FC/FI), thermospray ionization (TSP), nanospray ionization, for example. The masses are filtered and separated in the mass detector by several techniques that include, by way of illustration and not limitation, Time-of-Flight (TOF), ion traps, quadrupole mass filters, sector mass analysis, multiple reaction monitoring (MRM), and Fourier transform ion cyclotron resonance (FTICR), for example. The MS method detects the molecules using, for example, a microchannel plate, electron multiplier, or Faraday cup. The MS method can be repeated as a tandem MS/MS method, in which charged mass particles from a first MS are separated into a second MS. Pre-processing steps for separating molecules of interest, such as, by way of example, ambient ionization, liquid chromatography (LC), gas chromatography (GC), and affinity separation, can be used prior to the MS method.

Mass analyzers include, but are not limited to, quadrupoles, time-of-flight (TOF) analyzers, magnetic sectors, Fourier transform ion traps, and quadrupole ion traps, for example. Tandem (MS-MS) mass spectrometers are instruments that have more than one analyzer. Tandem mass spectrometers include, but are not limited to, quadrupole-quadrupole, magnetic sector-quadrupole, quadrupole-time-of-flight, for example. The detector of the mass spectrometer may be, by way of illustration and not limitation, a photomultiplier, an electron multiplier, or a micro-channel plate, for example.

### Examples of Affinity Agent

An affinity agent is a molecule capable of binding selectively to a rare molecule or a mass labels. Selective binding involves the specific recognition of one of two different molecules for the other compared to substantially less recognition of other molecules. The terms “binding” or “bound” refers to the manner in which two moieties are associated to one another.

An affinity agent can be a immunoglobulin, protein, peptide, metal, carbohydrate, metal chelator, nucleic acid or other molecule capable of binding selectively to a particular rare molecule or a mass labels type. Selective binding involves the specific recognition of one of two different molecules for the other compared to substantially less recognition of other molecules. The association is through non-covalent binding such as a specific ionic binding, hydrophobic binding, pocket binding and the like. In contrast, “non-specific binding” may result from several factors including hydrophobic or electrostatic interactions between molecules that are general and not specific to any particular molecule in a class of similar molecules.

The affinity agents which are immunoglobulins may include complete antibodies or fragment thereof, which immunoglobulins include the various classes and isotypes, such as IgA, IgD, IgE, IgG1, IgG2a, IgG2b and IgG3, IgM, etc. Fragments thereof may include Fab, Fv and F(ab′)₂, and Fab′, for example. In addition, aggregates, polymers, and conjugates of immunoglobulins or their fragments can be used where appropriate so long as binding affinity for a particular molecule is maintained.

Antibodies are specific for a rare molecule binding and can be monoclonal or polyclonal. Such antibodies can be prepared by techniques that are well known in the art such as immunization of a host and collection of sera (polyclonal) or by preparing continuous hybrid cell lines and collecting the secreted protein (monoclonal) or by cloning and expressing nucleotide sequences or mutagenized versions thereof coding at least for the amino acid sequences required for specific binding of natural antibodies.

Polyclonal antibodies and monoclonal antibodies may be prepared by techniques that are well known in the art. For example, in one approach monoclonal antibodies are obtained by somatic cell hybridization techniques. Monoclonal antibodies may be produced according to the standard techniques of Köhler and Milstein, Nature 265:495-497, 1975. Reviews of monoclonal antibody techniques are found in Lymphocyte Hybridomas, ed. Melchers, et al. Springer-Verlag (New York 1978), Nature 266: 495 (1977), Science 208: 692 (1980), and Methods of Enzymology 73 (Part B): 3-46 (1981). In general, monoclonal antibodies can be purified by known techniques such as, but not limited to, chromatography, e.g., DEAE chromatography, ABx chromatography, and HPLC chromatography; and filtration, for example.

An affinity agent can additionally be a “cell affinity agent” capable of binding selectively to a rare molecule which is used for typing a rare cell or measuring a biological intracellular process of a cell. These rare cell markers can be immunoglobulins that specifically recognizes and binds to an antigen associated with a particular cell type and whereby antigen are components of the cell. The cell affinity agent is capable of being absorbed into or onto the cell. The term “cell affinity agent” refers to a rare cell typing markers capable of binding selectively to rare cell. Selective cell binding typically involves “binding between molecules that is relatively dependent of specific structures of binding pair. Selective binding does not rely on non-specific recognition.

### Examples Label and Capture Particles

Affinity agent be attached to mass labels and/or particles for purpose of detection or isolation of rare molecules. This attachment can occur through “label particles” which are in turn attached mass labels. Affinity agents can also be attached to “capture particles” which allow separation of bound and unbound mass labels or rare molecule. This attachment to capture and label can be prepared by directly attached the affinity agent in a “linking group”. The terms “attached” or “attachment” refers to the manner in which two moieties are connected accomplished by a direct bond between the two moieties or a linking group between the two moieties. This allows the method to be multiplexed for more than one result at a time. Alternatively, affinity agent can be attached to mass labels and/or particles mass label using additional “binding partners”. The phrase “binding partner” refers to a molecule that is a member of a specific binding pair of affinity agent and “affinity tags” that bind each other and not the mass labels or rare molecules. In some cases, the affinity agent may be members of an immunological pair such as antigen to antibody or hapten to antibody, biotin to avidin, IgG to protein A, secondary antibody to primary antibody, antibodies to fluorescent labels and other examples binding pairs.

The “label particle” is a particulate material which can be attached to the affinity agent through a direct linker arm or a binding pair. Also the “label particle” is capable of forming an X—Y cleavable linkage between label particle and mass label. The size of the label particle is large enough to accommodate one or more mass label and affinity agent. The ratio of affinity agents or mass label to a single label particle may be 10⁷ to 1, 10⁶ to 1, or 10⁵ to 1, or 10⁴ to 1, or 10³ to 1, or 10² to 1, or 10 to 1, for example. The number of affinity agents and mass labels associated with the label particle is dependent on one or more of the nature and size of the affinity agent, the nature and size of the label particle, the nature of the linker arm, the number and type of functional groups on the label particle, and the number and type of functional groups on the Mass label, for example.

The composition of the label or capture particle entity may be organic or inorganic, magnetic or non-magnetic as a nanoparticle or a micro particle. Organic polymers include, by way of illustration and not limitation, nitrocellulose, cellulose acetate, poly(vinyl chloride), polyacrylamide, polyacrylate, polyethylene, polypropylene, poly(4-methylbutene), polystyrene, poly(methyl methacrylate), poly(hydroxyethyl methacrylate), poly(styrene/divinylbenzene), poly(styrene/acrylate), poly(ethylene terephthalate), demdrimer, melamine resin, nylon, poly(vinyl butyrate), for example, either used by themselves or in conjunction with other materials and including latex, microparticle and nanoparticle forms thereof. The particles may also comprise carbon (e.g., carbon nanotubes), metal (e.g., gold, silver, and iron, including metal oxides thereof), colloids, dendrimers, dendrons, and liposomes, for example. In some examples, the label particle may be a silica nanoparticle. In other some examples, label particles can be magnetic that have free carboxylic acid, amine or tosyl groups. In other some examples, label particles can be mesoporous and include mass labels inside the label particles.

The diameter of the label or capture particle is dependent on one or more of the nature of the rare molecule, the nature of the sample, the permeability of the cell, the size of the cell, the size of the nucleic acid, the size of the affinity agent, the magnetic forces applied for separation, the nature and the pore size of a filtration matrix, the adhesion of the particle to matrix, the surface of the particle, the surface of the matrix, the liquid ionic strength, liquid surface tension and components in the liquid, and the number, size, shape and molecular structure of associated label particles, for example.

The term “permeability” means the ability of a particles and molecule to enter a cell through the cell wall. In the case of detection of a rare molecule inside the cell, the diameter of the label particles must be small enough to allow the affinity agents to enter the cell. The label particle maybe coated with materials to increase “permeability” like collagenase, peptides, proteins, lipid, surfactants, and other chemicals known to increase particle inclusion into the cell.

When a porous matrix is employed in a filtration separation step, the diameter of the label particles must be small enough to be pass through the pores of a porous matrix if it did bind the rare molecule, and the diameter of the label particles must be large enough to not pass through the pores of a porous matrix to retain the bound rare molecule on the matrix. In some examples in accordance with the principles described herein, the average diameter of the label particles should be at least about 0.01 microns (10 nm) and not more than about 10 microns In some examples, the particles have an average diameter from about 0.02 microns to about 0.06 microns, or about 0.03 microns to about 0.1 microns, or about 0.06 microns to about 0.2 microns, or about 0.2 microns to about 1 micron, or about 1 micron to about 3 microns, or about 3 micron to about 10 microns. In some examples, the adhesion of the particles to the surface is so strong that the particle diameter can be smaller than the pore size of the matrix.

The affinity agent can be prepared by directly attached to the affinity agent to carrier or capture particles by linking groups. The linking group between the label particle and the affinity agent, may be aliphatic or aromatic bond. The linking groups may comprise a cleavable or non-cleavable linking moiety. Cleavage of the cleavable moiety can be the same achieved by electrochemical reduction used for the mass label but also may be achieved by chemical or physical methods, involving further oxidation, reduction, solvolysis, e.g., hydrolysis, photolysis, thermolysis, electrolysis, sonication, and chemical substitution, for example. Photocleavable bonds that are cleavable with light having an appropriate wavelength such as, e.g., UV light at 300 nm or greater; for example. The nature of the cleavage agent is dependent on the nature of the cleavable moiety. When heteroatoms are present, oxygen will normally be present as oxy or oxo, bonded to carbon, sulfur, nitrogen or phosphorous; sulfur will be present as thioether or thiono; nitrogen will normally be present as nitro, nitroso or amino, normally bonded to carbon, oxygen, sulfur or phosphorous; phosphorous will be bonded to carbon, sulfur, oxygen or nitrogen, usually as phosphonate and phosphate mono- or diester. Functionalities present in the linking group may include esters, thioesters, amides, thioamides, ethers, ureas, thioureas, guanidines, azo groups, thioethers, carboxylate and so forth. The linking group may also be a macro-molecule such as polysaccharides, peptides, proteins, nucleotides, and dendrimers.

The linking group between the particle and the affinity agent may be a chain of from 1 to about 60 or more atoms, or from 1 to about 50 atoms, or from 1 to about 40 atoms, or from 1 to 30 atoms, or from about 1 to about 20 atoms, or from about 1 to about 10 atoms, each independently selected from the group normally consisting of carbon, oxygen, sulfur, nitrogen, and phosphorous, usually carbon and oxygen. The number of heteroatoms in the linking group may range from about 0 to about 8, from about 1 to about 6, or about 2 to about 4. The atoms of the linking group may be substituted with atoms other than hydrogen such as, for example, one or more of carbon, oxygen and nitrogen in the form of, e.g., alkyl, aryl, aralkyl, hydroxyl, alkoxy, aryloxy, or aralkoxy groups. As a general rule, the length of a particular linking group can be selected arbitrarily to provide for convenience of synthesis with the proviso that there is minimal interference caused by the linking group with the ability of the linked molecules to perform their function related to the methods disclosed herein.

Obtaining reproducibility in amounts of particle captured after separation and isolation is important for rare molecular analysis. Additional, knowing the amounts of particle captured that enter a rare cell is important to maximize the amount of specific binding. Knowing the amount of particles remaining after washing are important to minimize the amount of non-selective binding.

In order to make these determination, it is helpful it the particles can contain fluorescent, optical or chemiluminescence labels. Therefore, label particles, can be measured by fluorescent or chemiluminescence by virtue of the presence of a fluorescent or chemiluminescence molecule. The fluorescent and optical molecule can then be measured by microscopic analysis and compared to expected results for sample containing and lacking analyte. Fluorescent molecule include but not limited to Dylight™, FITC, rhodamine compounds, phycoerythrin, phycocyanin, allophycocyanin, o-phthaldehyde, fluorescent rare earth chelates, amino-coumarins, umbelliferones, oxazines, Texas red, acridones, perylenes, indacines such as, e.g., 4,4-difluoro-4-bora-3a,4a-diaza-s-indacene and variants thereof, 9,10-bis-phenylethynyl-anthracene, squaraine dyes and fluorescamine, for example. A fluorescent microscope or fluorescent spectrometer may then be used to determine the location and amount of the label particles. Chemiluminescence labels examples include luminol, acridinium esters and acridinium sulfonamides to name a few. Optical labels examples include color particles, gold particles, enzymetic colorimetric reactions to name a few.

### Examples of Porous Matrix and Filtration

Porous matrices are used in examples to isolate capture particle and cells during the isolation and/or detection of rare molecule. Porous matrix are used where the particles are sufficiently smaller than the pore size of the matrix such that physically the particles can fall through the pores. In other examples, the particles are sufficiently larger than the pore size of the matrix such that physically the particles cannot fall through the pores.

In some methods in accordance with the principles described herein, the sample is incubated with an affinity agent comprised of a mass label and label particle, for each different population of rare molecules. The affinity agent that comprises a specific binding partner that is specific for and binds to a rare molecule of one of the populations of the rare molecules. The rare molecules can be cell bound or cell free. The affinity agent with mass label and label particle are retained on the surface of a membrane in a filtration.

The separation can occur in some examples when a porous matrix employed in filtration separation step is such that the pore diameter is smaller than the diameter of the cell with the rare molecule but larger than the unbound label particles to allow the affinity agents to achieve the benefits of rare molecule capture in accordance with the principles described herein but small enough to be pass through the pores of a porous matrix or matrix of a porous matrix if did not capture rare molecule. In other methods, the porous matrix employed in filtration separation step is such that the pore diameter is smaller than the diameter of the affinity agents on label particle capable of binding rare molecule but larger that the unbound molecule pass through allow the affinity agents to achieve the benefits of rare molecule capture. In still other methods, the affinity agents on label particle can be additionally bound through “binding partners” or sandwich assays to other capture particles, like magnetic particles, or to a surface, like a membrane. In the later case, the capture particles are retained on the surface of the porous membranes.

In all examples, the concentration of the one or more different populations of rare molecules is enhanced over that of the non-rare molecules to form a concentrated sample. In some examples, the sample is subjected to a filtration procedure using a porous matrix that retains the rare molecules while allowing the non-rare molecules to pass through the porous matrix thereby enhancing the concentration of the rare molecules. In the event that one or more rare molecules are non-cellular, i.e., not associated with a cell or other biological particle, the sample is combined with one or more capture particle entities wherein each capture particle entity comprises a binding partner for the non-cellular rare molecule of each of the populations of non-cellular rare molecules to render the non-cellular rare molecules in particulate form, i.e., to form particle-bound non-cellular rare molecules. The combination of the sample and the capture particle entities is held for a period of time and at a temperature to permit the binding of non-cellular rare molecules with corresponding binding partners of the capture particle entities. Vacuum is applied to the sample on the porous matrix to facilitate passage of non-rare cells and other particles through the matrix. The level of vacuum applied is dependent on one or more of the nature and size of the different populations of rare cells and/or particle reagents, the nature of the porous matrix, and the size of the pores of the porous matrix, for example.

Contact of the sample with the porous matrix is continued for a period of time sufficient to achieve retention of cellular rare molecules and/or particle-bound non-cellular rare molecules on a surface of the porous matrix to obtain a surface of the porous matrix having different populations of rare cells and/or particle-bound rare molecules as discussed above. The period of time is dependent on one or more of the nature and size of the different populations of rare cells and/or particle-bound rare molecules, the nature of the porous matrix, the size of the pores of the porous matrix, the level of vacuum applied to the blood sample on the porous matrix, the volume to be filtered, and the surface area of the porous matrix, for example. In some examples, the period of contact is about 1 minute to about 1 hour, about 5 minutes to about 1 hour, or about 5 minutes to about 45 minutes, or about 5 minutes to about 30 minutes, or about 5 minutes to about 20 minutes, or about 5 minutes to about 10 minutes, or about 10 minutes to about 1 hour, or about 10 minutes to about 45 minutes, or about 10 minutes to about 30 minutes, or about 10 minutes to about 20 minutes, for example.

An amount of each different affinity agent that is employed in the methods in accordance with the principles described herein is dependent on one or more of the nature and potential amount of each different population of rare molecules, the nature of the mass label, the natured of attachment, the nature of the affinity agent, the nature of a cell if present, the nature of a particle if employed, and the amount and nature of a blocking agent if employed, for example. In some examples, the amount of each different modified affinity agent employed is about 0.001 μg/μL to about 100 μg/μL, or about 0.001 μg/μL to about 80 μg/μL, or about 0.001 μg/μL to about 60 μg/μL, or about 0.001 μg/μL to about 40 μg/μL, or about 0.001 μg/μL to about 20 μg/μL, or about 0.001 μg/μL to about 10 μg/μL, or about 0.5 μg/μL to about 100 μg/μL, or about 0.5 μg/μL to about 80 μg/μL, or about 0.5 μg/μL to about 60 μg/μL, or about 0.5 μg/μL to about 40 μg/μL, or about 0.5 μg/μL to about 20 μg/μL, or about 0.5 μg/μL to about 10 μg/μL, for example.

The porous matrix is a solid, material, which is impermeable to liquid (except through one or more pores of the matrix is in accordance with the principles described herein. The porous matrix is associated with a porous matrix holder and a liquid holding well. The association between porous matrix and holder can be done with an adhesive. The association between porous matrix in the holder and the liquid holding well can be through direct contact or with a flexible gasket surface.

The porous matrix is a solid or semi-solid material and may be comprised of an organic or inorganic, water insoluble material. The porous matrix is non-bibulous, which means that the membrane is incapable of absorbing liquid. In some examples, the amount of liquid absorbed by the porous matrix is less than about 2% (by volume), or less than about 1%, or less than about 0.5%, or less than about 0.1%, or less than about 0.01%, or 0%. The porous matrix is non-fibrous, which means that the membrane is at least 95% free of fibers, or at least 99% free of fibers, or at least 99.5%, or at least 99.9% free of fibers, or 100% free of fibers.

The porous matrix can have any of a number of shapes such as, for example, track-etched, or planar or flat surface (e.g., strip, disk, film, matrix, and plate). The matrix may be fabricated from a wide variety of materials, which may be naturally occurring or synthetic, polymeric or non-polymeric. The shape of the porous matrix is dependent on one or more of the nature or shape of holder for the membrane, of the microfluidic surface, of the liquid holding well, of cover surface, for example. In some examples the shape of the porous matrix is circular, oval, rectangular, square, track-etched, planar or flat surface (e.g., strip, disk, film, membrane, and plate), for example.

The porous matrix and holder may be fabricated from a wide variety of materials, which may be naturally occurring or synthetic, polymeric or non-polymeric. Examples, by way of illustration and not limitation, of such materials for fabricating a porous matrix include plastics such as, for example, polycarbonate, poly (vinyl chloride), polyacrylamide, polyacrylate, polyethylene, polypropylene, poly-(4-methylbutene), polystyrene, polymethacrylate, poly(ethylene terephthalate), nylon, poly(vinyl butyrate), poly(chlorotrifluoroethylene), poly(vinyl butyrate), polyimide, polyurethane, and paraylene; silanes; silicon; silicon nitride; graphite; ceramic material (such, e.g., as alumina, zirconia, PZT, silicon carbide, aluminum nitride); metallic material (such as, e.g., gold, tantalum, tungsten, platinum, and aluminum); glass (such as, e.g., borosilicate, soda lime glass, and PYREX®); and bioresorbable polymers (such as, e.g., poly-lactic acid, polycaprolactone and polyglycoic acid); for example, either used by themselves or in conjunction with one another and/or with other materials. The material for fabrication of the porous matrix and holder are non-bibulous and does not include fibrous materials such as cellulose (including paper), nitrocellulose, cellulose acetate, rayon, diacetate, lignins, mineral fibers, fibrous proteins, collagens, synthetic fibers (such as nylons, dacron, olefin, acrylic, polyester fibers, for example) or, other fibrous materials (glass fiber, metallic fibers), which are bibulous and/or permeable and, thus, are not in accordance with the principles described herein. The material for fabrication of the porous matrix and holder may be the same or different materials.

The porous matrix for each liquid holding well comprises at least one pore and no more than about 2,000,000 pores per square centimeter (cm²). In some examples the number of pores of the porous matrix per cm² is 1 to about 2,000,000, or 1 to about 1,000,000, or 1 to about 500,000, or 1 to about 200,000, or 1 to about 100,000, or 1 to about 50,000, or 1 to about 25,000, or 1 to about 10,000, or 1 to about 5,000, or 1 to about 1,000, or 1 to about 500, or 1 to about 200, or 1 to about 100, or 1 to about 50, or 1 to about 20, or 1 to about 10, or 2 to about 500,000, or 2 to about 200,000, or 2 to about 100,000, or 2 to about 50,000, or 2 to about 25,000, or 2 to about 10,000, or 2 to about 5,000, or 2 to about 1,000, or 2 to about 500, or 2 to about 200, or 2 to about 100, or 2 to about 50, or 2 to about 20, or 2 to about 10, or 5 to about 200,000, or 5 to about 100,000, or 5 to about 50,000, or 5 to about 25,000, or 5 to about 10,000, or 5 to about 5,000, or 5 to about 1,000, or 5 to about 500, or 5 to about 200, or 5 to about 100, or 5 to about 50, or 5 to about 20, or 5 to about 10, for example. The density of pores in the porous matrix is about 1% to about 20%, or about 1% to about 10%, or about 1% to about 5%, or about 5% to about 20%, or about 5% to about 10%, for example, of the surface area of the porous matrix. In some examples, the size of the pores of a porous matrix is that which is sufficient to preferentially retain liquid while allowing the passage of liquid droplets formed in accordance with the principles described herein. The size of the pores of the porous matrix is dependent on the nature of the liquid, the size of the cell, the size of the capture particle, the size of mass label, the size of an analyte, the size of label particles, the size of non-rare molecules, and the size of non-rare cells, for example. In some examples the average size of the pores of the porous matrix is about 0.1 to about 20 microns, or about 0.1 to about 5 microns, or about 0.1 to about 1 micron, or about 1 to about 20 microns, or about 1 to about 5 microns, or about 1 to about 2 microns, or about 5 to about 20 microns, or about 5 to about 10 microns, for example.

Pores within the matrix may be fabricated in accordance with the principles described herein may be fabricated by, for example, microelectromechanical (MEMS) technology, metal oxide semiconductor (CMOS) technology, micro-manufacturing processes for producing microsieves, laser technology, irradiation, molding, and micromachining, for example, or a combination thereof.

The porous matrix is permanently fixed attached to a holder which can be associated to the bottom of the liquid holding well and to the top of the vacuum manifold where the porous matrix is positioned such that liquid can flow from liquid holding well to vacuum manifold. In some examples, the porous matrix in the holder can be associated to a microfluidic surface, top or bottom cover surface. The holder may be constructed of any suitable material that is compatible with the material of the porous matrix. Examples of such materials include, by way of example and not limitation, any of the materials listed above for the porous matrix. The material for the housing and for the porous matrix may be the same or may be different. The holder may also be constructed of non-porous glass or plastic film.

Examples of plastic film materials include polystyrene, polyalkylene, polyolefins, epoxies, Teflon®, PET, chloro-fluoroethylenes, polyvinylidene fluoride, PE-TFE, PE-CTFE, liquid crystal polymers, Mylar®, polyester, polymethylpentene, polyphenylene sulfide, and PVC plastic films. The plastic film can be metallized such as with aluminum. The plastic films can have relative low moisture transmission rate, e.g. 0.001 mg per m²-day. The porous matrix may be permanently fixed attached to a holder by adhesion using thermal bonding, mechanical fastening or through use of permanently adhesives such as drying adhesive like polyvinyl acetate, pressure-sensitive adhesives like acrylate-based polymers, contact adhesives like natural rubber and polychloroprene, hot melt adhesives like ethylene-vinyl acetates, and reactive adhesives like polyester, polyol, acrylic, epoxies, polyimides, silicones rubber-based and modified acrylate and polyurethane compositions, natural adhesive like dextrin, casein, lignin. The plastic film or the adhesive can be electrically conductive materials and the conductive material coatings or materials can be patterned across specific regions of the hold surface.

The porous matrix in the holder is generally part of a filtration module where the porous matrix is part of an assembly for convenient use during filtration. The holder does not contain pores and has a surface with facilitate contact with associated surfaces but is not permanently fixed attached to these surfaces and can be removed. A top gasket maybe applied to the removable holder between the liquid holding wells. A bottom gasket maybe applied to the removable holder between the manifold for vacuum. A gasket is a flexible material that facilities complete contact upon compression. The holder maybe constructed of gasket material. Examples of gasket shapes include a flat, embossed, patterned, or molded sheets, rings, circles, ovals, with cut out areas to allow sample to flow from porous matrix to vacuum maniford. Examples of gasket materials include paper, rubber, silicone, metal, cork, felt, neoprene, nitrile rubber, fiberglass, polytetrafluoroethylene like PTFE or Teflon or a plastic polymer like polychlorotrifluoroethylene.

In some examples, vacuum is applied to the concentrated and treated sample on the porous matrix to facilitate passage of non-rare cells through the matrix. The level of vacuum applied is dependent on one or more of the nature and size of the different populations of biological particles, the nature of the porous matrix, and the size of the pores of the porous matrix, for example. In some examples, the level of vacuum applied is about 1 millibar to about 100 millibar, or about 1 millibar to about 80 millibar, or about 1 millibar to about 50 millibar, or about 1 millibar to about 40 millibar, or about 1 millibar to about 30 millibar, or about 1 millibar to about 25 millibar, or about 1 millibar to about 20 millibar, or about 1 millibar to about 15 millibar, or about 1 millibar to about 10 millibar, or about 5 millibar to about 80 millibar, or about 5 millibar to about 50 millibar, or about 5 millibar to about 30 millibar, or about 5 millibar to about 25 millibar, or about 5 millibar to about 20 millibar, or about 5 millibar to about 15 millibar, or about 5 millibar to about 10 millibar, for example. In some examples the vacuum is an oscillating vacuum, which means that the vacuum is applied intermittently at regular of irregular intervals, which may be, for example, about 1 second to about 600 seconds, or about 1 second to about 500 seconds, or about 1 second to about 250 seconds, or about 1 second to about 100 seconds, or about 1 second to about 50 seconds, or about 10 seconds to about 600 seconds, or about 10 seconds to about 500 seconds, or about 10 seconds to about 250 seconds, or about 10 seconds to about 100 seconds, or about 10 seconds to about 50 seconds, or about 100 seconds to about 600 seconds, or about 100 seconds to about 500 seconds, or about 100 seconds to about 250 seconds, for example. In this approach, vacuum is oscillated at about 0 millibar to about 10 millibar, or about 1 millibar to about 10 millibar, or about 1 millibar to about 7.5 millibar, or about 1 millibar to about 5.0 millibar, or about 1 millibar to about 2.5 millibar, for example, during some or all of the application of vacuum to the blood sample. Oscillating vacuum is achieved using an on-off switch, for example, and may be conducted automatically or manually.

Contact of the treated sample with the porous matrix is continued for a period of time sufficient to achieve retention of the rare cells or the particle-bound rare molecules on a surface of the porous matrix to obtain a surface of the porous matrix having different populations of rare cells or the particle-bound rare molecules as discussed above. The period of time is dependent on one or more of the nature and size of the different populations of rare cells or particle-bound rare molecules, the nature of the porous matrix, the size of the pores of the porous matrix, the level of vacuum applied to the sample on the porous matrix, the volume to be filtered, and the surface area of the porous matrix, for example. In some examples, the period of contact is about 1 minute to about 1 hour, about 5 minutes to about 1 hour, or about 5 minutes to about 45 minutes, or about 5 minutes to about 30 minutes, or about 5 minutes to about 20 minutes, or about 5 minutes to about 10 minutes, or about 10 minutes to about 1 hour, or about 10 minutes to about 45 minutes, or about 10 minutes to about 30 minutes, or about 10 minutes to about 20 minutes, for example.

### Examples of Rare Molecules

The phrase “rare molecules” refers to a molecule that may be detected in a sample where the rare molecules is indicative of a particular population of molecules. The phrase “population of molecules” refers to a group of rare molecules that share a common rare molecules that is specific for the group of rare molecules. The phrase “specific for” means that the common rare molecules distinguishes the group of rare molecules from other molecules.

The methods described herein involve trace analysis, i.e., minute amounts of material on the order of 1 to about 100,000 copies of rare cells or rare molecules. Since this process involves trace analysis at the detection limits of the nucleic acid analyzers, these minute amounts of material can only be detected when detection volumes are extremely low, for example, 10⁻¹⁵ liter, so that the concentrations are within the detection. Given associated errors is unlikely and that “all” of the rare molecules undergo amplification, i.e., converting the minute amounts of material to the order of about 10⁵ to about 10¹⁰ copies of every rare molecule. The phrase “substantially all” means that at least about 70 to about 99% measured by the reproducibility of the amount of a rare molecule produced.

The phrase “cell free rare molecules” refers to rare molecules that are not bound to a cell and/or that freely circulate in a sample. Such non-cellular rare molecules include biomolecules useful in medical diagnosis of diseases, which include, but are not limited to, biomarkers for detection of cancer, cardiac damage, cardiovascular disease, neurological disease, hemostasis/hemastasis, fetal maternal assessment, fertility, bone status, hormone levels, vitamins, allergies, autoimmune diseases, hypertension, kidney disease, diabetes, liver diseases, infectious diseases and other biomolecules useful in medical diagnosis of diseases, for example.

The following are non-limiting examples of rare molecules that can be measured in a sample. The sample to be analyzed is one that is suspected of containing rare molecules. The samples may be biological samples or non-biological samples. Biological samples may be from a mammalian subject or a non-mammalian subject. Mammalian subjects may be, e.g., humans or other animal species. Biological samples include biological fluids such as whole blood, serum, plasma, sputum, lymphatic fluid, semen, vaginal mucus, feces, urine, spinal fluid, saliva, stool, cerebral spinal fluid, tears, and mucus, for example. Biological tissue includes, by way of illustration, hair, skin, sections or excised tissues from organs or other body parts, for example.

In many instances, the sample is whole blood, plasma or serum. Rare molecules may be from, for example, lung, bronchus, colon, rectum, pancreas, prostate, breast, liver, bile duct, bladder, ovary, brain, central nervous system, kidney, pelvis, uterine corpus, oral cavity or pharynx or melanoma cancers. The rare molecules may be from, but are not limited to, pathogens such as bacteria, virus, fungus, and protozoa; malignant cells such as malignant neoplasms or cancer cells; circulating endothelial cells; circulating tumor cells; circulating cancer stem cells; circulating cancer mesochymal cells; circulating epithelial cells; fetal cells; immune cells (B cells, T cells, macrophages, NK cells, monocytes); and stem cells; for example. In some examples of methods in accordance with the principles described herein, the sample to be tested is a blood sample from a mammal such as, but not limited to, a human subject, for example. The blood sample is one that contains cells such as, for example, non-rare cells and rare cells. In some examples the blood sample is whole blood or plasma.

Rare molecules of metabolic interest include but are not limited to those that impact the concentration of ACC Acetyl Coenzyme A Carboxylase, Adpn Adiponectin, AdipoR Adiponectin Receptor, AG Anhydroglucitol, AGE Advance glycation end products, Akt Protein kinase B, AMBK pre-alpha-1-microglobulin/bikunin, AMPK 5′-AMP activated protein kinase, ASP Acylation stimulating protein, Bik Bikunin, BNP B-type natriuretic peptide, CCL Chemokine (C—C motif) ligand, CINC Cytokine-induced neutrophil chemoattractant, CTF C-Terminal Fragment of Adiponectin Receptor, CRP C-reactive protein, DGAT Acyl CoA diacylglycerol transferase, DPP-IV Dipeptidyl peptidase-IV, EGF Epidermal growth factor, eNOS Endothelial NOS, EPO Erythropoietin, ET Endothelin, Erk Extracellular signal-regulated kinase, FABP Fatty acid-binding protein, FGF Fibroblast growth factor, FFA Free fatty acids, FXR Farnesoid X receptor a, GDF Growth differentiation factor, GH Growth hormone, GIP Glucose-dependent insulinotropic polypeptide, GLP Glucagon-like peptide-1, GSH Glutathione, GHSR Growth hormone secretagogue receptor, GULT Glucose transporters, GCD59 glycated CD59 (aka glyCD59), HbA1c Hemogloblin A1c, HDL High-density lipoprotein, HGF Hepatocyte growth factor, HIF Hypoxia-inducible factor, HMG 3-Hydroxy-3-methylglutaryl CoA reductase, I-α-I Inter-α-inhibitor, Ig-CTF Immunoglobulin attached C-Terminal Fragment of AdipoR, insulin, IDE Insulin-degrading enzyme, IGF Insulin-like growth factor, IGFBP IGF binding proteins, IL Interleukin cytokines, ICAM Intercellular adhesion molecule, JAK STAT Janus kinase/signal transducer and activator of transcription, JNK c-Jun N-terminal kinases, KIM Kidney injury molecule, LCN-2 Lipocalin, LDL Low-density lipoprotein, L-FABP Liver type fatty acid binding protein, LPS Lipopolysaccharide, Lp-PLA2 Lipoprotein-associated phospholipase A2, LXR Liver X receptors, LYVE Endothelial hyaluronan receptor, MAPK Mitogen-activated protein kinase, MCP Monocyte chemotactic protein, MDA Malondialdehyde, MIC Macrophage inhibitory cytokine, MIP Macrophage infammatory protein, MMP Matrix metalloproteinase, MPO Myeloperoxidase, mTOR Mammalian of rapamycin, NADH Nicotin-amide adenine dinucleotide, NGF Nerve growth factor, NFκB Nuclear factor kappa-light-chain-enhancer of activated B cells, NGAL Neutrophil gelatinase lipocalin, NOS Nitric oxide synthase NOX NADPH oxidase NPY Neuropeptide Yglucose, insulin, proinsulin, c peptide OHdG Hydroxydeoxyguanosine, oxLDL Oxidized low density lipoprotein, P-α-I pre-interleukin-α-inhibitor, PAI-1 Plasminogen activator inhibitor, PAR Protease-activated receptors, PDF Placental growth factor, PDGF Platelet-derived growth factor, PKA Protein kinase A, PKC Protein kinase C, PI3K Phosphatidylinositol 3-kinase, PLA2 Phosphatidylinositol 3-kinase, PLC Phospholipase C, PPAR Peroxisome proliferator-activated receptor, PPG Postprandial glucose, PS Phosphatidylserine, PR Proteinase, PYY Neuropeptide like peptide Y, RAGE Receptors for AGE, ROS Reactive oxygen species, 5100 Calgranulin, sCr Serum creatinine, SGLT2 Sodium-glucose transporter 2, SFRP4 secreted frizzled-related protein 4 precursor, SREBP Sterol regulatory element binding proteins, SMAD Sterile alpha motif domain-containing protein, SOD Superoxide dismutase, sTNFR Soluble TNF α receptor, TACE TNFα alpha cleavage protease, TFPI Tissue factor pathway inhibitor, TG Triglycerides, TGF β Transforming growth factorβ, TIMP Tissue inhibitor of metalloproteinases, TNFα Tumor necrosis factors α, TNFR TNF α receptor, THP Tamm-Horsfall protein, TLR Toll-like receptors, TnI Troponin I, tPA Tissue plasminogen activator, TSP Thrombospondin, Uri Uristatin, uTi Urinary trypsin inhibitor, uPA Urokinase-type plasminogen activator, uPAR uPA receptor, VCAM Vascular cell adhesion molecule, VEGF Vascular endothelial growth factor, and YKL-40 Chitinase-3-like protein.

Rare molecules of interest that are highly expressed by pancreas include but are not limited to INS insulin, GLU gluogen, NKX6-1 transcription factor, PNLIPRP1 pancreatic lipase-related protein 1, SYCN syncollin, PRSS1 protease, serine, 1 (trypsin 1) Intracellular, CTRB2 chymotrypsinogen B2 Intracellular, CELA2A chymotrypsin-like elastase family, member 2A, CTRB1 chymotrypsinogen B1 Intracellular, CELA3A chymotrypsin-like elastase family, member 3A Intracellular, CELA3B chymotrypsin-like elastase family, member 3B Intracellular, CTRC chymotrypsin C (caldecrin), CPA1 carboxypeptidase A1 (pancreatic) Intracellular, PNLIP pancreatic lipase, and CPB1 carboxypeptidase B1 (tissue), AMY2A amylase, alpha 2A (pancreatic), and CTFR cystic fibrosis transmembrane conductance regulator.

Rare molecules of interest that are highly expressed by adipose tissue include but are not limited to ADIPOQ Adiponectin, C1Q and collagen domain containing, TUSC5 Tumor suppressor candidate 5, LEP Leptin, CIDEA Cell death-inducing DFFA-like effector a, CIDEC Cell death-inducing DFFA-like effector C, FABP4 Fatty acid binding protein 4, adipocyte, LIPE, GYG2, PLIN1 Perilipin 1, PLIN4 Perilipin 4, CSN1S1, PNPLA2, RP11-407P15.2 Protein LOCl00509620, L GALS12 Lectin, galactoside-binding, soluble 12, GPAM Glycerol-3-phosphate acyltransferase, mitochondrial, PR325317.1 predicted protein, ACACB Acetyl-CoA carboxylase beta, ACVR1C Activin A receptor, type IC, AQP7 Aquaporin 7, CFD Complement factor D (adipsin)m CSN1S1Casein alpha sl, FASN Fatty acid synthase GYG2 Glycogenin 2 KIF25Kinesin family member 25 LIPELipase, hormone-sensitive PNPLA2 Patatin-like phospholipase domain containing 2 SLC29A4 Solute label family 29 (equilibrative nucleoside transporter), member 4 SLC7A10 Solute label family 7 (neutral amino acid transporter light chain, asc system), member 10, SPX Spexin hormone and TIMP4 TIMP metallopeptidase inhibitor 4.

Rare molecules of interest that are highly expressed by adrenal gland and thyroid include but are not limited to CYP11B2 Cytochrome P450, family 11, subfamily B, polypeptide 2, CYP11B1 Cytochrome P450, family 11, subfamily B, polypeptide 1, CYP17A1 Cytochrome P450, family 17, subfamily A, polypeptide 1, MC2R Melanocortin 2 receptor (adreno-corticotropic hormone), CYP21A2 Cytochrome P450, family 21, subfamily A, polypeptide 2, HSD3B2 Hydroxy-delta-5-steroid dehydrogenase, 3 beta- and steroid delta-isomerase 2, TH Tyrosine hydroxylase, AS3MT Arsenite methyltransferase, CYP11A1 Cytochrome P450, family 11, subfamily A, polypeptide 1, DBH Dopamine beta-hydroxylase (dopamine beta-monooxygenase), HSD3B2 Hydroxy-delta-5-steroid dehydrogenase, 3 beta- and steroid delta-isomerase 2, TH Tyrosine hydroxylase, AS3MT Arsenite methyltransferase, CYP11A1 Cytochrome P450, family 11, subfamily A, polypeptide 1, DBH Dopamine beta-hydroxylase (dopamine beta-monooxygenase), AKR1B1 Aldo-keto reductase family 1, member B1 (aldose reductase), NOV Nephroblastoma overexpressed, FDX1 Ferredoxin 1, DGKK Diacylglycerol kinase, kappa, MGARP Mitochondria-localized glutamic acid-rich protein, VWA5B2 Von Willebrand factor A domain containing 5B2, C18orf42 Chromosome 18 open reading frame 42, KIAA1024, MAP3K15 Mitogen-activated protein kinase kinase kinase 15, STAR Steroidogenic acute regulatory protein Potassium channel, subfamily K, member 2, NOV nephroblastoma overexpressed, PNMT phenylethanolamine N-methyltransferase, CHGB chromogranin B (secretogranin 1), and PHOX2A paired-like homeobox 2a.

Rare molecules of interest that are highly expressed by bone marrow include but are not limited to DEFA4 defensin alpha 4 corticostatin, PRTN3 proteinase 3, AZU1 azurocidin 1, DEFA1 defensin alpha 1, ELANE elastase, neutrophil expressed, DEFA1B defensin alpha 1B, DEFA3 defensin alpha 3 neutrophil-specific, MS4A3 membrane-spanning 4-domains, subfamily A, member 3 (hematopoietic cell-specific), RNASE3 ribonuclease RNase A family 3, MPO myeloperoxidase, HBD hemoglobin, delta, and PRSS57 protease, serine 57.

Rare molecules of interest that are highly expressed by the brain include but are not limited to GFAP glial fibrillary acidic protein, OPALIN oligodendrocytic myelin paranodal and inner loop protein, OLIG2 oligodendrocyte lineage transcription factor 2, GRIN1glutamate receptor ionotropic, N-methyl D-aspartate 1, OMG oligodendrocyte myelin glycoprotein, SLC17A7 solute label family 17 (vesicular glutamate transporter), member 7, Clorf6l chromosome 1 open reading frame 61, CREG2 cellular repressor of E1A-stimulated genes 2, NEUROD6 neuronal differentiation 6, ZDHHC22 zinc finger DHHC-type containing 22, VSTM2B V-set and transmembrane domain containing 2B, and PMP2 peripheral myelin protein 2.

Rare molecules of interest that are highly expressed by the endometrium, ovary, or placenta include but are not limited to MMP26 matrix metallopeptidase 26, MMP10 matrix metallopeptidase 10 (stromelysin 2), RP4-559A3.7 uncharacterized protein and TRH thyrotropin-releasing hormone.

Rare molecules of interest that are highly expressed by gastrointestinal tract, salivary gland, esophagus, stomach, duodenum, small intestine, or colon include but are not limited to GKN1 Gastrokine 1, GIF Gastric intrinsic factor (vitamin B synthesis), PGA5 Pepsinogen 5 group I (pepsinogen A), PGA3 Pepsinogen 3, group I (pepsinogen A, PGA4 Pepsinogen 4 group I (pepsinogen A), LCT Lactase, DEFA5 Defensin, alpha 5 Paneth cell-specific, CCL25 Chemokine (C—C motif) ligand 25, DEFA6 Defensin alpha 6 Paneth cell-specific, GAST Gastrin, MS4A10 Membrane-spanning 4-domains subfamily A member 10, ATP4A and ATPase, H+/K+ exchanging alpha polypeptide.

Rare molecules of interest that are highly expressed by heart or skeletal muscle include but are not limited to NPPB natriuretic peptide B, TNNI3 troponin I type 3 (cardiac), NPPA natriuretic peptide A, MYL7 myosin light chain 7 regulatory, MYBPC3 myosin binding protein C (cardiac), TNNT2 troponin T type 2 (cardiac) LRRC10 leucine rich repeat containing 10, ANKRD1 ankyrin repeat domain 1 (cardiac muscle), RD3L retinal degeneration 3-like, BMP10 bone morphogenetic protein 10, CHRNE cholinergic receptor nicotinic epsilon (muscle), and SBK2 SH3 domain binding kinase family member 2.

Rare molecules of interest that are highly expressed by kidney include but are not limited to UMOD uromodulin, TMEM174 transmembrane protein 174, SLC22A8 solute label family 22 (organic anion transporter) member 8, SLC12A1 solute label family 12 (sodium/potassium/chloride transporter) member 1, SLC34A1 solute label family 34 (type II sodium/phosphate transporter) member 1, SLC22A12 solute label family 22 (organic anion/urate transporter) member 12, SLC22A2 solute label family 22 (organic cation transporter) member 2, MCCD1 mitochondrial coiled-coil domain 1, AQP2 aquaporin 2 (collecting duct), SLC7A13 solute label family 7 (anionic amino acid transporter) member 13, KCNJ1 potassium inwardly-rectifying channel, subfamily J member 1 and SLC22A6 solute label family 22 (organic anion transporter) member 6.

Rare molecules of interest that are highly expressed by lung include but are not limited to SFTPC surfactant protein C, SFTPA1 surfactant protein A1, SFTPB surfactant protein B, SFTPA2 surfactant protein A2, AGER advanced glycosylation end product-specific receptor, SCGB3A2 secretoglobin family 3A member 2, SFTPD surfactant protein D, ROS1 proto-oncogene 1 receptor tyrosine kinase, MS4A15 membrane-spanning 4-domains subfamily A member 15, RTKN2 rhotekin 2, NAPSA napsin A aspartic peptidase, and LRRN4 leucine rich repeat neuronal 4.

Rare molecules of interest that are highly expressed by liver or gallbladder include but are not limited to APOA2 apolipoprotein A-II, A1BG alpha-1-B glycoprotein, AHSG alpha-2-HS-glycoprotein, F2coagulation factor II (thrombin), CFHR2 complement factor H-related 2, HPX hemopexin, F9 coagulation factor IX, CFHR2 complement factor H-related 2, SPP2 secreted phosphoprotein 2 (24 kDa), C9 complement component 9, MBL2 mannose-binding lectin (protein C) 2 soluble and CYP2A6 cytochrome P450 family 2 subfamily A polypeptide 6. Rare molecules of interest that are highly expressed by testis or prostate include but are not limited to PRM2 protamine 2 PRM1 protamine 1 TNP1 transition protein 1 (during histone to protamine replacement) TUBA3C tubulin, alpha 3c LELP1late cornified envelope-like proline-rich 1 BOD1L2 biorientation of chromosomes in cell division 1-like 2 ANKRD7 ankyrin repeat domain 7 PGK2 phosphoglycerate kinase 2 AKAP4 A kinase (PRKA) anchor protein 4 TPD52L3 tumor protein D52-like 3 UBQLN3 ubiquilin 3 and ACTL7A actin-like 7A.

### Examples of Rare Cells and Cell Markers

Rare cells are those cells that are present in a sample in relatively small quantities when compared to the amount of non-rare cells in a sample. In some examples, the rare cells are present in an amount of about 10⁻⁸% to about 10⁻²% by weight of a total cell population in a sample suspected of containing the rare cells. The phrase “cell rare molecules” refers to rare molecules that are bound in a cell and may or may not freely circulate in a sample. Such cellular rare molecule include biomolecules useful in medical diagnosis of diseases as above and also include all rare molecules and uses previously described in for cell free rare molecules and those for biomolecules used for measurement of rare cells. The rare cells (cell markers) may be, but are not limited to, and malignant cells such as malignant neoplasms or cancer cells; circulating cells, endothelial cells (CD146); epithelial cells (CD326/EpCAM); mesochymal cells (VIM), bacterial cells, virus, skin cells, sex cells, fetal cells; immune cells (leukocytes such as basophil, granulocytes (CD66b) and eosinophil, lymphocytes such as B cells (CD19, CD20), T cells (CD3, CD4 CD8), plasma cells, and NK cells (CD56), macrophages/monocytes (CD14, CD33), dendritic cells (CD11c, CD123), Treg cells and others), stem cells/precursor (CD34), other blood cells such as progenitor, blast, erythrocytes, thrombocytes, platelets (CD41, CD61, CD62) and immature cells; other cells from tissues such as liver, brain, pancreas, muscle, fat, lung, prostate, kidney, urinary tract, adipose, bone marrow, endometrium, gastrointestinal tract, heart, testis or other for example.

The phrase “population of cells” refers to a group of cells having an antigen or nucleic acid on their surface or inside the cell where the antigen is common to all of the cells of the group and where the antigen is specific for the group of cells. Non-rare cells are those cells that are present in relatively large amounts when compared to the amount of rare cells in a sample. In some examples, the non-rare cells are at least about 10 times, or at least about 10² times, or at least about 10³ times, or at least about 10⁴ times, or at least about 10⁵ times, or at least about 10⁶ times, or at least about 10⁷ times, or at least about 10⁸ times greater than the amount of the rare cells in the total cell population in a sample suspected of containing non-rare cells and rare cells. The non-rare cells may be, but are not limited to, white blood cells, platelets, and red blood cells, for example.

The term “Rare cells markers” include, but are not limited to, cancer cell type biomarkers, cancer bio markers, chemo resistance biomarkers, metastatic potential biomarkers, and cell typing markers, cluster of differentiation (cluster of designation or classification determinant) (often abbreviated as CD) is a protocol used for the identification and investigation of cell surface molecules providing targets for immunophenotyping of cells, for example. Cancer cell type biomarkers include, by way of illustration and not limitation, cytokeratins (CK) (CK1, CK2, CK3, CK4, CKS, CK6, CK7, CK8 and CK9, CK10, CK12, CK 13, CK14, CK16, CK17, CK18, CK19 and CK2), epithelial cell adhesion molecule (EpCAM), N-cadherin, E-cadherin and vimentin, for example. Oncoproteins and oncogenes with likely therapeutic relevance due to mutations include, but are not limited to, WAF, BAX-1, PDGF, JAGGED 1, NOTCH, VEGF, VEGHR, CAlX, MIB1, MDM, PR, ER, SELS, SEMI, PI3K, AKT2, TWIST1, EML-4, DRAFF, C-MET, ABL1, EGFR, GNAS, MLH1, RET, MEK1, AKT1, ERBB2, HER2, HNF1A, MPL, SMAD4, ALK, ERBB4, HRAS, NOTCH1, SMARCB1, APC, FBXW7, IDHL NPM1, SMO, ATM, FGFR1, JAK2, NRAS, SRC, BRAF, FGFR2, JAK3, RA, STK11, CDH1, FGFR3, KDR, PIK3CA, TP53, CDKN2A, FLT3, KIT, PTEN, VHL, CSF1R, GNA11, KRAS, PTPN11, DDR2, CTNNB1, GNAQ, MET, RB1, AKT1, BRAF, DDR2, MEK1, NRAS, FGFR1, and ROS1, for example.

In certain embodiments, the rare cells may be endothelial cells which are detected using markers, by way of illustration and not limitation, CD136, CD105/Endoglin, CD144/VE-cadherin, CD145, CD34, Cd41 CD136, CD34, CD90, CD31/PECAM-1, ESAM, VEGFR2/Fik-1, Tie-2, CD202b/TEK, CD56/NCAM, CD73/VAP-2, claudin 5, ZO-1, and vimentin. Metastatic potential biomarkers include, but are limited to, urokinase plasminogen activator (uPA), tissue plasminogen activator (tPA), C terminal fragment of adiponectin receptor (Adiponectin Receptor C Terminal Fragment or Adiponectin CTF), kinases (AKT-PIK3, MAPK), vascular adhesion molecules (e.g., ICAM, VCAM, E-selectin), cytokine signaling (TNF-α, IL-1, IL-6), reactive oxidative species (ROS), protease-activated receptors (PARs), metalloproteinases (TIMP), transforming growth factor (TGF), vascular endothelial growth factor (VEGF), endothelial hyaluronan receptor 1 (LYVE-1), hypoxia-inducible factor (HIF), growth hormone (GH), insulin-like growth factors (IGF), epidermal growth factor (EGF), placental growth factor (PDF), hepatocyte growth factor (HGF), nerve growth factor (NGF), platelet-derived growth factor (PDGF), growth differentiation factors (GDF), VEGF receptor (soluble Flt-1), microRNA (MiR-141), Cadherins (VE, N, E), S100 Ig-CTF nuclear receptors (e.g., PPARα), plasminogen activator inhibitor (PAI-1), CD95, serine proteases (e.g., plasmin and ADAM, for example); serine protease inhibitors (e.g., Bikunin); matrix metalloproteinases (e.g., MMP9); matrix metalloproteinase inhibitors (e.g., TIMP-1); and oxidative damage of DNA.

Chemoresistance biomarkers include, by way of illustration and not limitation, PL2L piwi like, 5T4, ADLH, β-integrin, α-6-integrin, c-kit, c-met, LIF-R, chemokines (e.g., CXCR7, CCR7, CXCR4), ESA, CD 20, CD44, CD133, CKS, TRAF2 and ABC transporters, cancer cells that lack CD45 or CD31 but contain CD34 are indicative of a cancer stem cell; and cancer cells that contain CD44 but lack CD24.

Rare cells of interest may be immune cells and include but are not limited to markers for white blood cells (WBC), Tregs (regulatory T cells), B cell, T cells, macrophages, monocytes, antigen presenting cells (APC), dendritic cells, eosinophils, and granulocytes. For example, markers such as, but not limited to, CD3, CD4, CD8, CD11c, CD14, CD15, CD16, CD19, CD20, CD31, CD33, CD45, CD52, CD56, CD 61, CD66b, CD123, CTLA-4, immunoglobulin, protein receptors and cytokine receptors and other CD marker that are present on white blood cells can be used to indicate that a cell is not a rare cell of interest. In a particular non-limiting example, CD45 antigen (also known as protein tyrosine phosphatase receptor type C or PTPRC) and originally called leukocyte common antigen is useful in detecting all white blood cells.

Additionally, CD45 can be used to differentiate different types of white blood cells that might be considered rare cells. For example, granulocytes are indicated by CD45+, CD15+, or CD16+, or CD66b+; monocytes are indicated by CD45+, CD14+; T lymphocytes are indicated by CD45+, CD3+; T helper cells are indicated by CD45+, CD3+, CD4+; cytotoxic T cells are indicated by CD45+, CD3+, CDS+; B-lymphocytes are indicated by CD45+, CD19+ or CD45+, CD20+; thrombocytes are indicated by CD45+, CD61+; and natural killer cells are indicated by CD16+, CD56+, and CD3-. Furthermore, two commonly used CD molecules, namely, CD4 and CD8, are, in general, used as markers for helper and cytotoxic T cells, respectively. These molecules are defined in combination with CD3+, as some other leukocytes also express these CD molecules (some macrophages express low levels of CD4; dendritic cells express high levels of CD11c, and CD123. These examples are not inclusive of all marker and are for example only.

In other cases the rare cell maybe a stem cell and include but are not limited to markers for stem cells including, PL2L piwi like, 5T4, ADLH, β-integrin, α6 integrin, c-kit, c-met, LIF-R, CXCR4, ESA, CD 20, CD44, CD133, CKS, TRAF2 and ABC transporters, cancer cells that lack CD45 or CD31 but contain CD34 are indicative of a cancer stem cell; and cancer cells that contain CD44 but lack CD24. Stem cell markers include common pluripotency markers like FoxD3, E-Ras, Sall4, Stat3, SUZ12, TCF3, TRA-1-60, CDX2, DDX4, Miwi, Mill GCNF, Oct4, Klf4, Sox2, c-Myc, TIF 1βPiwil, nestin, integrin, notch, AML, GATA, Esrrb, Nr5a2, C/EBPα, Lin28, Nanog, insulin, neuroD, adiponectin, apdiponectin receptor, FABP4, PPAR, and KLF4 and the like.

In other cases the rare cell maybe a pathogen, bacteria, or virus or group thereof which includes, but is not limited to, gram-positive bacteria (e.g., Enterococcus sp. Group B streptococcus, Coagulase-negative staphylococcus sp. Streptococcus viridans, Staphylococcus aureus and saprophyicus, Lactobacillus and resistant strains thereof, for example); yeasts including, but not limited to, Candida albicans, for example; gram-negative bacteria such as, but not limited to, Escherichia coli, Klebsiella pneumoniae, Citrobacter koseri, Citrobacter freundii, Klebsiella oxytoca, Morganella morganii, Pseudomonas aeruginosa, Proteus mirabilis, Serratia marcescens, Diphtheroids (gnb), Rosebura, Eubacterium hallii. Faecalibacterium prauznitzli, Lactobacillus gasseria, Streptococcus mutans, Bacteroides thetaiotaomicron, Prevotella Intermedia, Porphyromonas gingivalis Eubacterium rectale Lactobacillus amylovorus, Bacillus subtilis, Bifidobacterium longum Eubacterium rectale, E. eligens, E. dolichum, B. thetaiotaomicron, E. rectale, Actinobacteria, Proteobacteria, B. thetaiotaomicron, Bacteroides Eubacterium dolichum, Vulgatus, B. fragilis, bacterial phyla such as Firmicuties (Clostridia, Bacilli, Mollicutes), Fusobacteria, Actinobacteria, Cyanobacteria, Bacteroidetes, Archaea, Proteobacteria, and resistant strains thereof, for example; viruses such as, but not limited to, HIV, HPV, Flu, and MERSA, for example; and sexually transmitted diseases. In the case of detecting rare cell pathogens, a particle reagent is added that comprises a binding partner, which binds to the rare cell pathogen population. Additionally, for each population of cellular rare molecules on the pathogen, a reagent is added that comprises a binding partner for the cellular rare molecule, which binds to the cellular rare molecules in the population.

As mentioned above, some examples in accordance with the principles described herein are directed to methods of detecting a cell, which include natural and synthetic cells. The cells are usually from a biological sample that is suspected of containing target rare molecules, non-rare cells and rare cells. The samples may be biological samples or non-biological samples. Biological samples may be from a mammalian subject or a non-mammalian subject. Mammalian subjects may be, e.g., humans or other animal species.

Biological samples include biological fluids such as whole blood, serum, plasma, sputum, lymphatic fluid, semen, vaginal mucus, feces, urine, spinal fluid, saliva, stool, cerebral spinal fluid, tears, and mucus, for example. Biological tissue includes, by way of illustration, hair, skin, sections or excised tissues from organs or other body parts, for example. In many instances, the sample is whole blood, plasma or serum. Rare cells may be from, for example, lung, bronchus, colon, rectum, pancreas, prostate, breast, liver, bile duct, bladder, ovary, brain, central nervous system, kidney, pelvis, uterine corpus, oral cavity or pharynx or melanoma cancers. In some examples of methods in accordance with the principles described herein, the sample to be tested is a blood sample from a mammal such as, but not limited to, a human subject, for example. The blood sample is one that contains cells such as, for example, non-rare cells and rare cells. In some examples the blood sample is whole blood or plasma.

### Kits for Conducting Methods

The apparatus and reagents for conducting a method in accordance with the principles described herein may be present in a kit useful for conveniently performing the method. In one embodiment, a kit comprises in packaged combination modified affinity agent one for each different rare molecule acid to be isolated. The kit may also comprise one or more, cell affinity agent to for cell containing the rare molecules, the porous matrix, optional capture particles, and solution for spraying, filtering and reacting the mass labels. The composition of the label particle may be, for example, as described above for capture particle entities. Porous matrix and electrode can be in housing where the house can have vents, capillaries, chambers, liquid inlets and outlets. A solvent is applied to a membrane and can be a spray liquid. Porous matrix can be removable.

Depending on method for analysis of rare molecules selected, reagents discussed in more detail herein below, may or may not be used to treat the samples during, prior or after the extract molecules from the rare cells and cell free samples.

The relative amounts of the various reagents in the kits can be varied widely to provide for concentrations of the reagents that substantially optimize the reactions that need to occur during the present methods and further to optimize substantially the sensitivity of the methods. Under appropriate circumstances one or more of the reagents in the kit can be provided as a dry powder, usually lyophilized, including excipients, which on dissolution will provide for a reagent solution having the appropriate concentrations for performing a method in accordance with the principles described herein. The kit can further include a written description of a method utilizing reagents in accordance with the principles described herein.

The phrase “at least” as used herein means that the number of specified items may be equal to or greater than the number recited. The phrase “about” as used herein means that the number recited may differ by plus or minus 10%; for example, “about 5” means a range of 4.5 to 5.5.

The spray solvent can be any spray solvent employed in electrospray mass spectroscopy. In some examples, solvents for electrospray ionization include, but are not limited to, polar organic compounds such as, e.g., alcohols (e.g., methanol, ethanol and propanol), acetonitrile, dichloromethane, dichloroethane, tetrahydrofuran, dimethylformamide, dimethyl sulphoxide, and nitromethane; non-polar organic compounds such as, e.g., hexane, toluene, cyclohexane; and water, for example, or combinations of two or more thereof. Optionally, the solvents may contain one or more of an acid or a base as a modifier (such as, volatile salts and buffer, e.g., ammonium acetate, ammonium biocarbonate, volatile acids such as formic acid, acetic acids or trifluoro-acetic acid, heptafluorobutyric acid, sodium dodecyl sulphate, ethylenediamine tetraacetic acid, and non-volatile salts or buffers such as, e.g., chlorides and phosphates of sodium and potassium, for example.

In many examples, the above mentioned spray solvents might be used in combination with aqueous medium, which may be solely water or which may also contain organic solvents such as, for example, polar aprotic solvents, polar protic solvents such as, e.g., dimethylsulfoxide (DMSO), dimethylformamide (DMF), acetonitrile, an organic acid, or an alcohol, and non-polar solvents miscible with water such as, e.g., dioxane, in an amount of about 0.1% to about 50%, or about 1% to about 50%, or about 5% to about 50%, or about 1% to about 40%, or about 1% to about 30%, or about 1% to about 20%, or about 1% to about 10%, or about 5% to about 40%, or about 5% to about 30%, or about 5% to about 20%, or about 5% to about 10%, by volume. In some examples, the pH for the aqueous medium is usually a moderate pH. In some examples, the pH of the aqueous medium is about 5 to about 8, or about 6 to about 8, or about 7 to about 8, or about 5 to about 7, or about 6 to about 7, or physiological pH. Various buffers may be used to achieve the desired pH and maintain the pH during any incubation period. Illustrative buffers include, but are not limited to, borate, phosphate (e.g., phosphate buffered saline), carbonate, TRIS, barbital, PIPES, HEPES, MES, ACES, MOPS, and BICINE.

Cell lysis reagents are those that involve disruption of the integrity of the cellular membrane with a lytic agent, thereby releasing intracellular contents of the cells. Numerous lytic agents are known in the art. Lytic agents that may be employed may be physical and/or chemical agents. Physical lytic agents include, blending, grinding, and sonication, and combinations or two or more thereof, for example. Chemical lytic agents include, but are not limited to, non-ionic detergents, anionic detergents, amphoteric detergents, low ionic strength aqueous solutions (hypotonic solutions), bacterial agents, and antibodies that cause complement dependent lysis, and combinations of two or more thereof, for example, and combinations or two or more of the above. Non-ionic detergents that may be employed as the lytic agent include both synthetic detergents and natural detergents.

The nature and amount or concentration of lytic agent employed depends on the nature of the cells, the nature of the cellular contents, the nature of the analysis to be carried out, and the nature of the lytic agent, for example. The amount of the lytic agent is at least sufficient to cause lysis of cells to release contents of the cells. In some examples the amount of the lytic agent is (percentages are by weight) about 0.0001% to about 0.5%, about 0.001% to about 0.4%, about 0.01% to about 0.3%, about 0.01% to about 0.2%, about 0.1% to about 0.3%, about 0.2% to about 0.5%, about 0.1% to about 0.2%, for example.

Removal of lipids may be carried out using, by way of illustration and not limitation, detergents, surfactants, solvents, and binding agents, and combinations of two or more of the above, for example, and combinations of two or more thereof. The use of a surfactant or a detergent as a lytic agent as discussed above accomplishes both cell lysis and removal of lipids. The amount of the agent for removing lipids is at least sufficient to remove at least about 50%, or at least about 60%, or at least about 70%, or at least about 80%, or at least about 90%, or at least about 95% of lipids from the cellular membrane. In some examples the amount of the lytic agent is (percentages by weight) about 0.0001% to about 0.5%, about 0.001% to about 0.4%, about 0.01% to about 0.3%, about 0.01% to about 0.2%, about 0.1% to about 0.3%, about 0.2% to about 0.5%, about 0.1% to about 0.2%, for example.

In some examples, it may be desirable to remove or denature proteins from the cells, which may be accomplished using a proteolytic agent such as, but not limited to, proteases, heat, acids, phenols, and guanidinium salts, and combinations of two or more thereof, for example. The amount of the proteolytic agent is at least sufficient to degrade at least about 50%, or at least about 60%, or at least about 70%, or at least about 80%, or at least about 90%, or at least about 95% of proteins in the cells. In some examples the amount of the lytic agent is (percentages by weight) about 0.0001% to about 0.5%, about 0.001% to about 0.4%, about 0.01% to about 0.3%, about 0.01% to about 0.2%, about 0.1% to about 0.3%, about 0.2% to about 0.5%, about 0.1% to about 0.2%, for example.

In some examples, samples are collected from the body of a subject into a suitable container such as, but not limited to, a cup, a bag, a bottle, capillary, or a needle, for example. Blood samples may be collected into VACUTAINER® containers, for example. The container may contain a collection medium into which the sample is delivered. The collection medium is usually a dry medium and may comprise an amount of platelet deactivation agent effective to achieve deactivation of platelets in the blood sample when mixed with the blood sample.

Platelet deactivation agents can be added to the sample such as, but are not limited to, chelating agents such as, for example, chelating agents that comprise a triacetic acid moiety or a salt thereof, a tetraacetic acid moiety or a salt thereof, a pentaacetic acid moiety or a salt thereof, or a hexaacetic acid moiety or a salt thereof. In some examples, the chelating agent is ethylene diamine tetraacetic acid (EDTA) and its salts or ethylene glycol tetraacetate (EGTA) and its salts. The effective amount of platelet deactivation agent is dependent on one or more of the nature of the platelet deactivation agent, the nature of the blood sample, level of platelet activation and ionic strength, for example. In some examples, for EDTA as the anti-platelet agent, the amount of dry EDTA in the container is that which will produce a concentration of about 1.0 to about 2.0 mg/mL of blood, or about 1.5 mg/mL of the blood. The amount of the platelet deactivation agent is that which is sufficient to achieve at least about 90%, or at least about 95%, or at least about 99% of platelet deactivation.

Moderate temperatures are normally employed, which may range from about 5° C. to about 70° C. or from about 15° C. to about 70° C. or from about 20° C. to about 45° C., for example. The time period for an incubation period is about 0.2 seconds to about 6 hours, or about 2 seconds to about 1 hour, or about 1 to about 5 minutes, for example.

In many examples, the above combination is provided in an aqueous medium, which may be solely water or which may also contain organic solvents such as, for example, polar aprotic solvents, polar protic solvents such as, e.g., dimethylsulfoxide (DMSO), dimethylformamide (DMF), acetonitrile, an organic acid, or an alcohol, and non-polar solvents miscible with water such as, e.g., dioxane, in an amount of about 0.1% to about 50%, or about 1% to about 50%, or about 5% to about 50%, or about 1% to about 40%, or about 1% to about 30%, or about 1% to about 20%, or about 1% to about 10%, or about 5% to about 40%, or about 5% to about 30%, or about 5% to about 20%, or about 5% to about 10%, by volume. In some examples, the pH for the aqueous medium is usually a moderate pH. In some examples the pH of the aqueous medium is about 5 to about 8, or about 6 to about 8, or about 7 to about 8, or about 5 to about 7, or about 6 to about 7, or physiological pH, for example. Various buffers may be used to achieve the desired pH and maintain the pH during any incubation period. Illustrative buffers include, but are not limited to, borate, phosphate (e.g., phosphate buffered saline), carbonate, TRIS, barbital, PIPES, HEPES, MES, ACES, MOPS, and BICINE, for example.

An amount of aqueous medium employed is dependent on a number of factors such as, but not limited to, the nature and amount of the sample, the nature and amount of the reagents, the stability of rare cells, and the stability of rare molecules, for example. In some examples in accordance with the principles described herein, the amount of aqueous medium per 10 mL of sample is about 5 mL to about 100 mL, or about 5 mL to about 80 mL, or about 5 mL to about 60 mL, or about 5 mL to about 50 mL, or about 5 mL to about 30 mL, or about 5 mL to about 20 mL, or about 5 mL to about 10 mL, or about 10 mL to about 100 mL, or about 10 mL to about 80 mL, or about 10 mL to about 60 mL, or about 10 mL to about 50 mL, or about 10 mL to about 30 mL, or about 10 mL to about 20 mL, or about 20 mL to about 100 mL, or about 20 mL to about 80 mL, or about 20 mL to about 60 mL, or about 20 mL to about 50 mL, or about 20 mL to about 30 mL, for example.

Where one or more of the rare nucleic acids are part of a cell, the aqueous medium may also comprise a lysing agent for lysing of cells. A lysing agent is a compound or mixture of compounds that disrupt the integrity of the matrix of cells thereby releasing intracellular contents of the cells. Examples of lysing agents include, but are not limited to, non-ionic detergents, anionic detergents, amphoteric detergents, low ionic strength aqueous solutions (hypotonic solutions), bacterial agents, aliphatic aldehydes, and antibodies that cause complement dependent lysis, for example. Various ancillary materials may be present in the dilution medium. All of the materials in the aqueous medium are present in a concentration or amount sufficient to achieve the desired effect or function.

In some examples, it may be desirable to fix the nucleic acids or cells of the sample. Fixation immobilizes the nucleic acids and preserves the nucleic acids structure and maintains the cells in a condition that closely resembles the cells in an in vivo-like condition and one in which the antigens of interest are able to be recognized by a specific affinity agent. The amount of fixative employed is that which preserves the nucleic acids or cells but does not lead to erroneous results in a subsequent assay. The amount of fixative depends on one or more of the nature of the fixative and the nature of the cells, for example. In some examples, the amount of fixative is about 0.05% to about 0.15% or about 0.05% to about 0.10%, or about 0.10% to about 0.15%, for example, by weight. Agents for carrying out fixation of the cells include, but are not limited to, cross-linking agents such as, for example, an aldehyde reagent (such as, e.g., formaldehyde, glutaraldehyde, and paraformaldehyde); an alcohol (such as, e.g., C₁-C₅ alcohols such as methanol, ethanol and isopropanol); a ketone (such as a C₃-C₅ ketone such as acetone); for example. The designations C₁-C₅ or C₃-C₅ refer to the number of carbon atoms in the alcohol or ketone. One or more washing steps may be carried out on the fixed cells using a buffered aqueous medium.

In examples in which fixation is employed, extraction of nucleic acids can include a procedure for de-fixation prior to amplification. De-fixation may be accomplished employing, by way of illustration and not limitation, heat or chemicals capable of reversing cross-linking bonds, or a combination of both, for example.

In some examples utilizing the techniques, it may be necessary to subject the rare cells to permeabilization. Permeabilization provides access through the cell membrane to nucleic acids of interest. The amount of permeabilization agent employed is that which disrupts the cell membrane and permits access to the nucleic acids. The amount of permeabilization agent depends on one or more of the nature of the permeabilization agent and the nature and amount of the rare cells, for example. In some examples, the amount of permeabilization agent by weight is about 0.1% to about 0.5%, or about 0.1% to about 0.4%, or about 0.1% to about 0.3%, or about 0.1% to about 0.2%, or about 0.2% to about 0.5%, or about 0.2% to about 0.4%, or about 0.2% to about 0.3%, for example. Agents for carrying out permeabilization of the rare cells include, but are not limited to, an alcohol (such as, e.g., C₁-C₅ alcohols such as methanol and ethanol); a ketone (such as a C₃-C₅ ketone such as acetone); a detergent (such as, e.g., saponin, Triton® X-100, and Tween®-20); for example. One or more washing steps may be carried out on the permeabilized cells using a buffered aqueous medium.

The following examples further describe the specific embodiments of the invention by way of illustration and not limitation and are intended to describe and not to limit the scope of the invention. Parts and percentages disclosed herein are by volume unless otherwise indicated.

### EXAMPLES

All chemicals may be purchased from the Sigma-Aldrich Company (St. Louis Mo.) unless otherwise noted.

### Abbreviations:

min=minute(s)
μm=micron(s)
mL=milliliter(s)
mg=milligrams(s)
μg=microgram(s)
TE buffer=10 mM Tris-HCl, 1 mM EDTA, pH 7.5.
Proteolytic buffer=25 mM Tris-NaCl, 0.3% proteinase K
Saline-sodium citrate buffer (SSC)=3 M NaCl, 0.3 M sodium citrate, pH 7
SKBR cells=SKBR3 human breast cancer cells (ATCC)
WBC=white blood cells
Silica amine label particle=Propylamine-functionalized silica nano-particles 200 μm, mesoporous pore sized 4 nm
Porous Matrix=WHATMAN® NUCLEOPORE™ Track Etch matrix, 25 mm diameter and 8.0 and 1.0 μM pore sizes

### Example 1

### Release of Mass Tag from Rare Molecule Thin Layer Electrochemical Reactor Cell

A porous matrix was used which was a non-absorbent membrane of polycarbonate that was flexible and had about 100,000 pores of 8 μm diameter in a 22 mm diameter circle. The membrane was placed in a thin layered reaction chamber. The thin layer electrochemical reactor cell consisted of a titanium-based working electrode for reduction, a titanium auxiliary (counter) electrode, and a Pd/H₂ reference electrode was constructed according to FIG. 6. A 100-μm spacer was used to separate the working electrode and the auxiliary electrode inlet block giving a cell volume of approximately 6 μL. An electrical grounding union was used to decouple the electrochemical cell from the ESI high voltage.

An affinity agent with a mass label was added to the membrane in sufficient concentration to be detected was added to the membrane. A few μL of spray solvent was added. Spray solvent used was 1% Formic acid (250 mmol/L) in water with 5% acetonitrile. Gradient, potential was applied in waved E1, E2: −1.5, +1 V, t1, t2: 1990, 1010 ms, square wave pulse schematic representation of the square-wave pulse. Under optimized conditions, the potentials were −1.5 V (E1) and +1.0 V (E2) and time intervals were 1,990 ms (t1) and 1,010 ms (t2), unless specified otherwise up to 4000 mV, positive and negative ions more.

The electrospray ionization (ESI) was then caused with the electric field strength at a solvent-air interface ample in magnitude to overcome the forces due to surface tension of the liquid. At this point, the liquid is drawn into a cone from which charged droplets were expelled. These droplets underwent evaporation and fission cycles to ultimately produce gas-phase ions that were drawn into the vacuum system of a mass spectrometer for analysis. In the generation of an electrospray directly from the membrane surface in this example, the solution to be sprayed must sufficiently wet the top-side of the chip (etched silicon wafer housing) which contains the microwells.

A solvent which displays ideal wettability with the surface will inherently fill the wells upon solvent addition, thus providing a capillary flow for continuous solvent delivery during spray events. The back side of the emitter plate should ideally have a non-wetting interaction with the spray solvent. This type of interaction isolates the liquid to single drops on each of the 5-μm pores. The presence of individual droplets creates a high degree of curvature (compared to a flat, wetted surface) which produces greater electric field strength under the application of an electric potential, thus aiding in the formation of an electrospray. Additionally, by positioning the capillary inlet of a mass spectrometer in close proximity to the bottom side of the membrane, electric field strength is further enhanced and allows the generation of the electrospray from selected regions of the membrane, thus recovering spatial information. The spray solvent should be sufficiently polar and have a surface tension low enough to permit electrospray at electric field strengths lower than those which produce electrical breakdown of air.

Experiments were performed in which the standard straight capillary for the atmospheric pressure inlet (API) of a THERMO LTQ (linear ion trap) mass spectrometer (from Thermo Electron North America LLC) was replaced with an extended capillary which was bent at a 90° angle, such that the opening was pointing up. In all experiments, the membrane was positioned with the bottom side parallel to the ground approximately 1 mm distant from the bent capillary inlet. The bottom side of the membrane and the bent capillary were illuminated using a diode laser and video was recorded with a CMOS camera.

In the first set of experiments, 5 μL of spray solvent with release chemical TCEP was pipetted directly onto the top side of the membrane and potential was applied by directing the plasma from an antistatic gun towards the solvent. When potential was applied in this manner, discrete spray events were visualized and recorded mass spectra showed peaks typical of spraying the same solution via nanoESI. Upon the depletion of solvent, spectra were drastically different and were characteristic of those seen when firing the antistatic gun unobstructed at the MS inlet.

Further experiments showed that a porous membrane will spray but the amount of material sprayed is variable (20% cv) and the time at which the ejection of analyte ions and charged droplets containing analyte occurs is variable as well by more than 100 msec making the trapping of ions difficult as it occurs over short time scales and with small spray volumes; thus, resulting in a method that is not quantitative. As a control, an impermeable layer having pores of fixed orientation and being flexible (causing angle of greater than 1 degree) was tested. The result with this layer was that material sprayed varied by more than 20% CV. The membrane employed having characteristics in accordance with the principles described herein yielded spray amounts that were less than 1% CV.

A comparison of the performance of electrochemical release and chemical release membranes was made by spraying a mass label from both the membrane exposed to reduction and membranes exposed to chemical release agent. This comparison was made using membranes that had previously been used to filter blood. In the case of the flexible membrane, the membrane was positioned approximately 0.5 mm above the bent MS inlet and a 6.5 kV potential was applied to a wire (the electric field generator) positioned approximately 5 mm above the membrane.

### Example 2

### Capture of Mass Tag with Rare Molecule in Thin Layer Electrochemical Reactor Cell

A polypeptide with cysteine was used as rare molecule and SDPD nanopartiles particles used as the mass label was and capture particle. Both were added to the membrane in sufficient concentration for polycysteine to be measured and a few μL of solvent added. Spray solvent used was water with 5% acetonitrile. Gradient, potential for oxidation was applied in waved E1, E2: +1 V, −1.5, t1, t2: 1990, 1010 ms, square wave pulse schematic representation of the square-wave pulse. Under optimized conditions, the potentials were 1.0 V (E1) and −1.0 V (E2) and time intervals were 1,990 ms (t1) and 1,010 ms (t2), unless specified otherwise up to 4000 mV, positive and negative ion more. Experiments were performed using a membrane with and without electrochemical cell and measured using for the atmospheric pressure inlet (API) of a THERMO LTQ (linear ion trap) mass spectrometer (from Thermo Electron North America LLC) as above.

In the first set of experiments, the amount of oxidation coupling was measured as a function of time. Spray solvent was pipetted directly onto the top side of the membrane and polycysteine unbound measured. This showed advantage of porous matrix in a electrochemical cell, where complete and 1 seconds completion of binding occurred with membrane in chamber and little to no binding occurred without electrochemical cell or no matrix at 5 min. Some binding with electrochemical cell or no matrix occurred at 5 min. The electrochemical chamber and matrix is used and the reaction rate is msec vs minute half live with chemical attachment complete. The capture peptide was released as in above example 1 and still showed an advantage of reagent free release in example 1, with no reducing agents, no reversibility or loss, and no suppression in acid and 10× more sensitive to allow without TCEP.

All patents, patent applications and publications cited in this application including all cited references in those patents, applications and publications, are hereby incorporated by reference in their entirety for all purposes to the same extent as if each individual patent, patent application or publication were so individually denoted.

While the many embodiments of the invention have been disclosed above and include presently preferred embodiments, many other embodiments and variations are possible within the scope of the present disclosure and in the appended claims that follow. Accordingly, the details of the preferred embodiments and examples provided are not to be construed as limiting. It is to be understood that the terms used herein are merely descriptive rather than limiting and that various changes, numerous equivalents may be made without departing from the spirit or scope of the claimed invention.

### REFERENCES

1. Karen A. Sap and Jeroen A. A. Demmers (2012). Labeling Methods in Mass Spectrometry Based Quantitative Proteomics, Integrative Proteomics, Dr. Hon-Chiu Leung (Ed.), ISBN: 978-953-51-0070-6, InTech, Available from: http://www.intechopen.com/books/integrative-proteomics/labeling-methods-in-mass-spectrometry-basedquantitative-proteomics
2. Thompson, A., Schafer, J., Kuhn, K., Kienle, S., Schwarz, J., Schmidt, G., Neumann, T., Johnstone, R., Mohammed, A. K. & Hamon, C. (2003). Tandem Mass Tags: A Novel Quantification Strategy for Comparative Analysis of Complex Protein Mixtures by Ms/Ms. Anal Chem, 75(8), 1895-1904.
3. Ross, P. L., Huang, Y. N., Marchese, J. N., Williamson, B., Parker, K., Hattan, S., Khainovski, N., Pillai, S., Dey, S., Daniels, S., Purkayastha, S., Juhasz, P., Martin, S., Bartle twww.intechopen.com Labeling Methods in Mass Spectrometry Based Quantitative Proteomics 131
4. Jones, M., He, F., Jacobson, A. & Pappin, D. J. (2004). Multiplexed Protein Quantitation in Saccharomyces Cerevisiae Using Amine-Reactive Isobaric Tagging Reagents. Mol Cell Proteomics, 3(12), 1154-1169.
5. Magbanua M J, Pugia M, Lee J S, Jabon M, Wang V, Gubens M, Marfurt K, Pence J, Sidhu H, Uzgiris A, Rugo H S, Park J W. (2015) A Novel Strategy for Detection and Enumeration of Circulating Rare Cell Populations in Metastatic Cancer Patients Using Automated Microfluidic Filtration and Multiplex Immunoassay. PLoS One. 10(10):e0141166. PMID:26496203. PMCID:PMC4619669
6. Baird Z, Pirro V, Ayrton S, Hollerbach A, Hanau C, Marfurt K, Foltz M, Cooks R G, Pugia M. (2015) Tumor Cell Detection by Mass Spectrometry Using Signal Ion Emission Reactive Release Amplification. M. Anal Chem 2016 Jul. 19; 88(14):6971-5. PMID:27351295
7. Robert Popp⋅David Malmström⋅Andrew G Chambers, D. Lin, A. G Camenzind, J Grace van der Gugten, D. T. Holmes, M. Pugia, M. Jaremek, S Cornett, D. Suckau, C H Borchers AAn Automated Assay for the Clinical Measurement of Plasma Renin Activity by immuno-MALDI (iMALDI). Biochimica et Biophysica Acta—Proteins & Proteomics 10/; 1854(6) (2014).
8. Y. Zhu, R. Valdes Jr., C. Q. Simmons, M. W. Linder, M. J. Pugia, S. A. Jortani. Analysis of Ligand binding by Bioaffinity Mass Spectrometry. Clin Chem Acta 371(1-2), 71-8 (2006).
9. Dmitry R. Bandura, Vladimir I. Baranov, Olga I. Ornatsky, Alexei Antonov, Robert Kinach, Xudong Lou, Serguei Pavlov, Sergey Vorobiev, John E. Dick, and Scott D. Tanner. Mass Cytometry: Technique for Real Time Single Cell Multitarget Immunoassay Based on Inductively Coupled Plasma Time-of-Flight Mass Spectrometry. Anal. Chem. 2009, 81, 6813-6822
10. Jung Rok Lee, Juhee Lee, Sang Kyung Kim, Kwang Pyo Kim, Hyung Soon Park, Woon-Seok Yeo. Mass Spectrometry Signal Amplification Method for Attomolar Detection of Antigens Using Small-Molecule-Tagged Gold Microparticles Angew. Chem. Int. Ed. 2008, 47, 9518-9521
11. Suming Chen, Qiongqiong Wan, Abraham K. Badu-Tawiah. Mass Spectrometry for Paper-Based Immunoassays: Toward On□Demand Diagnosis. J. Am. Chem. Soc. 2016, 138, 6356-6359
12. James P. Tam, Cui Rong Wu, Wen Liu, Jing Wen Zhangm Disulfide bond formation in peptides by dimethyl sulfoxide. Scope and applications J. Am. Chem. Soc., 1991, 113 (17), pp 6657-6662
13. Colin Thorpe Quantification of Thiols and Disulfides Biochim Biophys Acta. 2014 February; 1840(2)
14. Claus Jacob. Metal and Redox Modulation of Cysteine Protein Function Chemistry & Biology, Vol. 10, 677-693, August, 2003
15. Renee L. Pasker Overview of Affinity Tags for Protein Purification Curr Protoc Protein Sci.; 73: Unit-9.9
16. J. E. Hale Irreversible, Oriented Immobilization of Antibodies to Cobalt-Iminodiacetate Resin for Use as Immunoaffinity Analytical Biochemistry, Volume 231, Issue 1, October 1995, Pages 46-49
17. France Arnold. Review: Multipoint binding and heterogeneity in immobilized metal affinity chromatography Biotechnology and Bioengineering 48 1995, Pages 437-443
18. Commonly owned pending U.S. application Ser. No. 15/941,059 entitled Methods And Apparatus For Removal Of Small Volume From A Filtration Device filed Mar. 30, 2018 and Ser. No. 15/941,125 entitled Methods And Apparatus For Selective Nucleic Acid Analysis filed Mar. 30, 2018 all incorporated by reference herein.

What is claimed is:

1. A method for the attachment or release of mass labels, said method comprising attaching or releasing said mass labels by electrochemical reduction or electrochemical oxidation.

2. The method of claim 1, wherein bond breakage or bond formation of mass labels occurs by said electrochemical reactions in a porous matrix placed between an anode and cathode electrode in a thin layer electrochemical reactor.

3. The method of claim 2, wherein said attachment or release of said mass labels by electrochemical reduction or oxidation occurs by breaking or a forming an —X—Y— bond where X and Y can be a S, O, C, N or a metal.

4. The method of claim 3, wherein the metal is selected from the group consisting of Mn, Fe, Co, Ni, Cu, Zn, Mo, Tc, Ru Rh, Pd, Ag Cd, In, Sn, Ir, Pt Au, Hg, Ti and Pb.

5. The method of claim 4, wherein said mass labels are attached to an affinity agent for a rare molecule by an —X—Y— bond.

6. The method of claim 4, wherein said mass labels are separated with an affinity agent for a rare molecule.

7. The method of claim 4, wherein said mass labels are released by electrochemical reduction breaking of the —X—Y— bond.

8. The method of claim 4, wherein said mass labels are measured and related to rare molecule.

9. The method of claim 5, wherein said mass labels are attached to a particle.

10. The method of claim 9, wherein the particle is an organic amine particle.

11. The method of claim 9, wherein fluorescent, chemiluminescence or optical labels are attached to the same particle.

12. The method of claim 5, wherein the rare molecule is additionally bonded to an affinity agent attached to a particle.

13. The method of claim 5, wherein the affinity agents attached to a particle are separated by magnetic or filtration on to a porous matrix.

14. The method of claim 5, wherein the rare molecule is a peptide, protein or molecule capable of being bound by an affinity agent.

15. The method of claim 1, wherein said mass labels are attached to a rare molecule via an —X—Y— bond by electrochemical oxidation.

16. The method of claim 12, wherein mass labels are attached to a particle.

17. The method of claim 16, where the particle is an organic amine particle.

18. The method of claim 17, wherein fluorescent, chemiluminescence or optical labels are attached to the same particle.

19. The method of claim 14, where the rare molecule is additionally bonded to an affinity agent attached to a particle.

20. The method of claim 14, wherein the rare molecule is modified to form an —X—Y-bond.
